SEQUENCE LISTING (6 sequences)

SEQ ID NO: 1 - DNA, 20 bp, Mus musculus
  source 1..20: mol_type = other DNA
cccctatcacaattagcatt

SEQ ID NO: 2 - DNA, 18 bp, Homo sapiens
  source 1..18: mol_type = other DNA
cctatcacgattagcatt

SEQ ID NO: 3 - DNA, 20 bp, synthetic construct
  source 1..20: mol_type = other DNA
ccagggcaagatgagggaga

SEQ ID NO: 4 - DNA, 20 bp, synthetic construct
  source 1..20: mol_type = other DNA
ggacctcggttggcaatgta

SEQ ID NO: 5 - DNA, 24 bp, synthetic construct
  source 1..24: mol_type = other DNA
tgatggcgtggacagtggtcataa

SEQ ID NO: 6 - DNA, 24 bp, synthetic construct
  source 1..24: mol_type = other DNA
catgtttgtgatgggcgtgaacca

(19) United States
(12) Patent Application Publication
(10) Pub. No.: US 2024/0415804 A1
(43) Pub. Date: Dec. 19, 2024

(54) METHOD OF TREATING PANCREATIC CANCER

(71) Applicant: University of South Florida, Tampa, FL (US)

(72) Inventor: Tomar Ghansah, Riverview, FL (US)

(21) Appl. No.: 18/824,458

(22) Filed: Sep. 4, 2024

Related U.S. Application Data
(63) Continuation of application No. PCT/US2023/015201, filed on Mar. 14, 2023.
(60) Provisional application No. 63/357,940, filed on Jul. 1, 2022.
(60) Provisional application No. 63/269,278, filed on Mar. 14, 2022.

(51) Int. Cl. A61K 31/352; A61P 35/00; C12N 15/113

(52) U.S. Cl. CPC A61K 31/352; A61P 35/00; C12N 15/113; C12N 2310/141

(57) ABSTRACT

A novel method of treating cancer is presented. API transcriptionally regulates SHIP-1 expression via the suppression of miRNA-155, impacting anti-tumor immune responses in the bone marrow (BM) and TME of mice with PC. The inventors discovered that API reduced miRNA-155 in the PC milieu, which induced SHIP-1 expression. This promoted the restoration of myelopoiesis and increased anti-tumor immune responses in the TME of heterotopic, orthotopic and transgenic SHIP-1 knockout preclinical mouse models of PC. The results suggest that manipulating SHIP-1 through miR-155 can assist in augmenting anti-tumor immune responses and aid in the therapeutic intervention of PC. Further, administration of both API and an miR-155 inhibitor were found to act synergistically to treat pancreatic cancer.

### CROSS REFERENCE TO RELATED APPLICATIONS

This application is a continuation of and claims priority to International Application Serial No. PCT/US2023/015201, entitled “Method of Treating Pancreatic Cancer”, filed Mar. 14, 2023, which claims priority to U.S. Provisional Patent Application Ser. No. 63/269,278, entitled “SHIP-1 Regulation of Tumoricidal Macrophages and Neutrophils that Increase Anti-Tumor Responses in Pancreatic Cancer”, filed Mar. 14, 2022, and U.S. Provisional Application Ser. No. 63/357,940, entitled “Apigenin Targets MicroRNA-155, Enhances SHIP-1 Expression, Improves Myelopoiesis and Anti-Tumor Responses in Pancreatic Cancer”, filed Jul. 1, 2022, the contents of each of which are hereby incorporated by reference into this disclosure.

### SEQUENCE LISTING

The sequence listing entitled “Method of Treating Pancreatic Cancer” in XML format, created on Aug. 22, 2023 and being 7000 bytes in size, is hereby incorporated by reference into this disclosure.

### FIELD OF INVENTION

This invention relates to treatment of cancers. More specifically, the present invention provides therapeutic methods for treating pancreatic cancer.

### BACKGROUND OF THE INVENTION

Pancreatic cancer (PC) is an aggressive and lethal malignancy. Based on its increasing incidence it is projected to become the second leading cause of cancer-related death after lung cancer in the US by 20301-4. The lethality is attributed to late diagnosis, early metastasis, and limited response to current chemotherapies: gemcitabine, nab-paclitaxel and FOLFIRINOX⁵. Despite advances in immunotherapy for the treatment of multiple solid tumors, the role of immunotherapy in PC remains limited because inflammatory tumor-derived factors (TDF) contribute to the induction of an immunosuppressive tumor microenvironment (TME). This immunosuppressive TME inactivates anti-tumor immune responses by halting the recruitment of effector immune cells into the tumor⁶⁻⁸. The inflammatory TME does this, in part, by inducing the expansion of regulatory immunosuppressive myeloid-derived suppressor cells (MDSC) generated from the bone marrow (BM) and found in human PC and pre-clinical PC models. These MDSC consist of immature myeloid cells, macrophages, granulocytes and dendritic cells (DC) that aggressively suppress anti-tumor immunity via multiple modalities⁶⁻⁹. Furthermore, expansion of MDSCs has also been reported in the BM and tumors of PC patients compared with healthy controls¹⁰ and these MDSC have been reported, in pre-clinical models of PC, to be responsible for the suppression of anti-tumor immune responses^{11, 12}. In addition, granulocytic and monocytic MDSC (M-MDSC), mobilized into the TME and M-MDSC, can differentiate into protumor M2 tumor-associated macrophages (M2-TAM) vs. tumoricidal M1 TAM, which have the potential to further suppress anti-tumor immune responses, and promote metastasis and chemoresistance in PC¹³⁻¹⁶.

MicroRNAs (miRs) are small, nonprotein-coding RNAs (18-24 nucleotides in length) that can inhibit gene expression at the post-transcriptional level through binding to the complementary sequences of their target mRNAs at 30-untranslated regions (30-UTRs)¹⁷. They have been shown to regulate biological processes such as cell proliferation, cellular differentiation, stem cell development, homeostasis and apoptosis, consequently affecting biological events such as cell survival, immune modulation and carcinogenesis¹⁷⁻²⁰. Deregulation of miRNAs has been associated with almost all human malignancies, either acting as oncogenes (OncoMirs) or tumor suppressors²¹. One of the first miRNAs identified with oncogenic potential was miR-155 which was found to be overexpressed in lymphoma, breast, colon and PC^{21, 22}. MiR-155 presented the highest prognostic impact in PC patients linked to poor survival²³⁻²⁵. MIR-155 targets Src Homology-2 (SH2) domain-containing Inositol 50-Phosphatase-1 (SHIP-1) transcription and thus regulates inflammation, MDSC activation and polarization of TAM²⁶⁻²⁸.

SHIP-1 is a 145 kDa protein that regulates the activity of macrophages²⁹⁻³². SHIP-1 expression is regulated in immune cells by external soluble factors such as cytokines and chemokines in the microenvironment³³. The inventors have shown that SHIP-1 knockout (KO) mice have markedly increased numbers of immunosuppressive, protumor M2 macrophages, demonstrating the role of SHIP-1 in regulating macrophage polarization^{12, 29, 34}. In addition, we have reported that downregulation of SHIP-1 protein expression and expansion of MDSC corresponds with an increase in tumor burden in mice with PC^{11, 35}. Therefore, suppression of miR-155 production may promote upregulation of SHIP-1 expression and thus restore M-MDSC homeostasis, increase tumoricidal M1-TAM percentages and improve anti-tumor immune responses in PC.

Several studies have suggested that increased intake of dietary fruits, vegetables, and cereal grains may prevent gastrointestinal cancers, including PC³⁶⁻³⁸. Recently, natural compounds known as bioflavonoids including apigenin (API) have been demonstrated in both in vitro and in vivo models to exert broad anticancer activities in a variety of malignancies such as breast cancer³⁹, liver cancer⁴⁰, prostate cancer⁴¹, lung cancer⁴², colon cancer⁴³, melanoma⁴⁴, osteosarcoma⁴⁵ and PC^{12, 46, 47}. API inhibits tumor cell by inducing apoptosis leading to autophagy and cell cycle arrest at the G2/M phase and can also reduce cancer cell motility, thereby preventing cancer cell migration and invasion regulating PI3K/AKT, MAPK/ERK, JAK/STAT, NF-κB, p53 and Wnt/B-catenin signaling pathways^{48, 49}. API has demonstrated potent anti-tumor activity and the ability to reduce chemoresistance to gemcitabine (one of the chemotherapy drugs used for PC) in human PC cell lines⁵⁰. API has also been shown to induce apoptosis through p53-dependent and p53-independent mechanisms⁵¹. The induction of apoptosis induced by API involves both extrinsic and intrinsic pathways in cancer cells^{52, 53}. Apoptosis targets of API consist of caspase-3, -8, and -9, Bax, Bak, Bad, Bim, Bid, Bcl-XL, XIAP, Mcl-1, Bcl-2, m-TOR/PI3K/AKT, STAT3, p53, p21, p27, PARP cleavage, FOXO3a, AIF, Apaf-1, DR5, ERK/JNK/p38 MAPK, Jun, NF-κB, Noxa, PUMA, Smac, Survivin, FAS and TRAILS1. API has shown the most selective killing of cancer cells while sparing normal cells⁵⁴. The inventors recently reported that API reduced tumor burden, improved anti-tumor immune responses and increased survival rates of mice bearing pancreatic tumors compared with vehicle treated mice with PC^{12, 55}. However, the ability of API to target miR-155 and SHIP-1 expression in PC has not been studied. The inventors have found that it targets miR-155, enhancing SHIP-1 expression, which leads to a restoration of MDSC homeostasis, and an increase in tumoricidal M1-TAM percentages thereby improving anti-tumor immune responses in mice with PC.

### SUMMARY OF THE INVENTION

Pancreatic cancer (PC) is one of the most lethal cancers with a grim prognosis. Pancreatic tumor derived factors (TDF) contribute to the induction of an immunosuppressive tumor microenvironment (TME) that impedes the effectiveness of immunotherapy. Further, PC promotes the expansion of immunosuppressive Myeloid-Derived Suppressor Cells (MDSC) and Tumor Associated Macrophages (TAM) that dampen anti-tumor immunity and renders immunotherapies ineffective. PC-induced microRNA-155 (miRNA-155) represses expression of Src homology 2 (SH2) domain-containing Inositol 50-phosphatase-1 (SHIP-1), a regulator of myeloid cell development and function, thus impacting anti-tumor immunity.

The inventors recently reported that the bioflavonoid apigenin (API) increased SHIP-1 expression which correlated with the expansion of tumoricidal macrophages (TAM) and improved anti-tumor immune responses in the TME of mice with PC. API was shown to promote the development of monocytic-MDSC (M-MDSC) into M1 TAM (Tumoricidal) in the pancreatic tumor microenvironment (TME), which corresponded with an increase in anti-tumor immunity (tumor regression) mice harboring PC.

The inventors have now discovered that API transcriptionally regulates SHIP-1 expression via the suppression of miRNA-155, impacting anti-tumor immune responses in the bone marrow (BM) and TME of mice with PC. The inventors discovered that API reduced miRNA-155 in the PC milieu, which induced SHIP-1 expression. This promoted the restoration of myelopoiesis and increased anti-tumor immune responses in the TME of heterotopic, orthotopic and transgenic SHIP-1 knockout preclinical mouse models of PC. The results suggest that manipulating SHIP-1 through miR-155 can assist in augmenting anti-tumor immune responses and aid in the therapeutic intervention of PC.

In an embodiment, a method of treating pancreatic cancer is presented comprising: administering, to the patient, a therapeutically effective amount of a therapeutic agent capable of increasing expression of SH-2 containing inositol 5′ polyphosphatase 1 (SHIP 1) and administering, to the patient, a therapeutically effective amount of a therapeutic agent capable of inhibiting expression of miR-155 wherein administration of both of the therapeutic agents acts synergistically to treat the pancreatic cancer by augmenting anti-tumor immune responses.

The therapeutic agent capable of increasing expression of SHIP 1 may be apigenin. The therapeutic agents may be administered concomitantly. The therapeutic agents may be administered at least 3 times per week.

In an embodiment, method of inducing cancer cell death is presented comprising: administering a therapeutically effective amount of a therapeutic agent capable of increasing expression of SH-2 containing inositol 5′ polyphosphatase 1 (SHIP 1) wherein administration of the therapeutic agent increases apoptosis and reduces the cancer cell viability.

The therapeutic agent capable of increasing expression of SHIP 1 may be apigenin. The method may further comprise administering a therapeutically effective amount of a therapeutic agent capable of inhibiting expression of miR-155. The administration of both of the therapeutic agents acts synergistically to induce cell death. The therapeutically effective agents may be administered concomitantly at least 3 times per week.

In an embodiment, a method of determining prognosis of and treating pancreatic cancer in a patient in need thereof comprising: measuring or having measured an expression level of an miR-155 biomarker in a sample from the patient; comparing or having compared the expression level of the miR-155 from the patient to a control sample wherein a higher differential expression of the miR-155 biomarker from the patient as compared to the control sample is indicative of a poor prognosis for the patient; administering, to the patient, a therapeutically effective amount of a therapeutic agent capable of increasing expression of SH-2 containing inositol 5′ polyphosphatase 1 (SHIP 1). The therapeutic agent capable of increasing expression of SHIP 1 may be apigenin.

The method may further comprise administering, to the patient, a therapeutically effective amount of a therapeutic agent capable of inhibiting expression of miR-155 wherein administration of both of the therapeutic agents acts synergistically to treat the pancreatic cancer. The therapeutic agents may be administered concomitantly.

The method may further comprise measuring or having measured an expression level of SH-2 containing inositol 5′ polyphosphatase 1 (SHIP 1) in a sample from the patient; and comparing or having compared the expression level of the SHIP 1 from the patient to a control sample wherein a lower differential expression of the SHIP 1 from the patient as compared to the control sample is indicative of a poor prognosis for the patient. The steps may be performed prior to administration of either of the therapeutic agents.

In an embodiment, a method of monitoring neoplasia progression from one biological state to another in a tumor sample is presented comprising: obtaining or having obtained an expression level of miR-155 from a sample from the patient at a first timepoint; obtaining or having obtained an expression level of miR-155 from a sample from the patient at a second timepoint, wherein the second timepoint is after the first timepoint; comparing the two expression levels to each other, wherein an increase in the expression level at the second timepoint as compared to the first timepoint is indicative of neoplasia progression and a decrease in the expression level at the second timepoint as compared to the first timepoint is indicative of neoplasia regression. The neoplasia may be pancreatic cancer.

In a further embodiment, a method of determining efficacy of a pancreatic cancer treatment is provided comprising: obtaining or having obtained an expression level of miR-155 from a sample from the patient at a first timepoint; administering a therapeutically effective amount of a therapeutic agent capable of increasing expression of SH-2 containing inositol 5′ polyphosphatase 1 (SHIP 1), such as apigenin; obtaining or having obtained an expression level of miR-155 from a sample from the patient at a second timepoint, wherein the second timepoint is after the first timepoint; comparing the two expression levels to each other, wherein an increase in the expression level at the second timepoint as compared to the first timepoint is indicative of a non-efficacious treatment and a decrease in the expression level at the second timepoint as compared to the first timepoint is indicative of efficacious treatment. Optionally, a therapeutically effective amount of a therapeutic agent capable of inhibiting expression of miR-155 may also be administered to the patient prior to measuring the expression level at the second timepoint. The administration of both of the therapeutic agents acts synergistically to treat the pancreatic cancer. The therapeutic agents may be administered concomitantly.

### BRIEF DESCRIPTION OF THE DRAWINGS

For a fuller understanding of the invention, reference should be made to the following detailed description, taken in connection with the accompanying drawings, in which:

FIG. 1A-B is an image depicting the proposed model: (A) In the PC tumor microenvironment, tumor derived factors such as PC-induced miRNA-155 impacts hematopoietic stem cells (HSC) and alters myelopoiesis by targeting and downregulating SHIP-1 gene and protein expressions. The reduction in SHIP-1 expression corresponds with the expansion of M-MDSC that correspond with the development of pro-tumor M2-TAM which, in turn, impair anti-tumor immunity and result in PC progression and metastasis. (B) The therapeutic use of the bioflavonoid API induces apoptosis and necrosis of PC cells and causes a decrease in miRNA-155 in the pancreatic tumor. This reduction correlates with an increase in SHIP-1 gene and protein expressions that results in the expansion of M-MDSC which coincides with the development of tumoricidal M1-TAM. These tumoricidal M1-TAM have increased CD40 expression which interacts with CD40L on CD8+ T cells, resulting in M1-TAM activation, production of iNOS/NO and an overall increase in anti-tumor immunity. This is signified by the release of granzyme B (GrB) and perforin (PRF) as well as the robust activation of effector CD8+ T cells, leading to PC regression.

FIG. 2A-F are a series of graphs depicting API suppressed miR-155 gene expression and inhibited cell viability in PC cells in vitro. The indicated human and mouse PC cell lines were treated with API (40 μM) or vehicle (CTRL) for 24 h and assessed for (A, C, E) miR-155 gene expression by qPCR or (B, D, F) cell viability by MTT assay. Data presented as the mean±S.D. of each experimental group (n=3). *p<0.05; **p<0.001; p<0.001 (by two-tailed t test).

FIG. 3A-D are as series of images depicting API induced apoptosis in murine PC cell lines. Flow cytometric analysis of apoptosis (AnnexinV+PI+) of (A.) Panc02 and (B.) UN-KC-6141 cells were treated with API (10-50 μM). Statistic were done by comparing to CTRL group. (C. and D.) WB analysis and quantification of anti-apoptotic protein, Bcl-2, in UN-KC-6141 cells treated with API (40 μM). Data are represented as the mean±S.D. of each experimental group (n=3) *p<0.05; **p≤0.01; ***p<0.001 (by two-tailed t test).

FIG. 4A-F are a series of graphs depicting Inhibition of miR-155 expression decreased the viability of PC cells in vitro. The indicated human and mouse PC cells were treated with miR-155 inhibitor or scrambled miRNA (100 nM) for 24 h and assessed for (A, C, E) miR-155 gene expression by qPCR or (B, D, F) cell viability by MTT assay. Data presented as the mean±S.D. of each experimental group (n=3). *p<0.05; ***p<0.001 (by two-tailed/test).

FIG. 5A-B are a series of graphs depicting miR-155 gene expression and cell viability are synergistically suppressed in murine UN-KC-6141 cells after combined treatment with API and miR-155 inhibitor. UN-KC-6141 cells were treated with miR-155 inhibitor (100 nM), API (40 μM), their respective controls or in combination and assessed for (A) miR-155 gene expression or (B) cell viability. Data are presented as the mean±S.D. of each experimental group (n=3). *p<0.05; **p<0.001; ***p<0.001; ****p<0.0001 (by two-tailed t test).

FIG. 6A-D are a series of images depicting API decreased miR-155 expression, which correlates with an increase in SHIP-1 expression in HPC mice. Relative (A.) miR-155 and (B.) SHIP-1 gene expression in the tumors of HPC and API-treated HPC mice. (C. and D.) Western blot analysis and representative quantification of PI-treated HPC mice. Data are represented as the mean±S.D. of HPC (n=3-4), HPC-API (n=3-4). *p<0.05; ****p<0.0001 (by two-tailed/test).

FIG. 7A-E are a series of images depicting API treatment decreased miR-155 expression in OPC mice, which correlated with increased SHIP-1 expression. Relative (A) miR-155 and (B) SHIP-1 gene expression in the BM of CTRL, OPC and OPC-API mice. (C, D) WB analysis and representative quantification of SHIP-1 protein in the BM of CTRL, OPC and OPC-API mice. (E) Relative miR-155 gene expression in the pancreas or tumor of CTRL, OPC and OPC-API mice. Data are presented as the mean±S.D. of CTRL (n=3), OPC (n=3-4), OPC-API (n=3-4) mice. *p<0.05; **p<0.001; ****p<0.0001 (by two-tailed/test).

FIG. 8A-D are a series of images depicting API treatment decreased miR-155 expression in KC-HPC mice, which correlated with an increase in SHIP-1 expression. Relative (A) miR-155 and (B) SHIP-1 gene expression in the tumors from KC—HPC and KC-HPC-API mice. (C, D) WB analysis and representative quantification of SHIP-1 protein from the tumors of KC-HPC and KC-HPC-API mice. Data are presented as the mean±S.D. of KC-HPC (n=3-4), KC-HPC-API (n=3-4). *p<0.05; **p<0.01 (by two-tailed t test).

FIG. 9A-B are a series of images depicting miR-155 gene expression is increased in the BM and Tumor of SHIP^{KO}-HPC mice. Relative quantification of miR-155 gene expression in the (A) BM and (B) tumors from SHIP^{WT}-HPC and SHIP^{KO}-HPC mice. Data are presented as the mean±S.D. SHIP^{KO}-HPC (n=3) and SHIP^{WT}-HPC (n=3) mice. *p<0.05 (by two-tailed t test).

FIG. 10A-B are a series of images depicting API treatment of KC-HPC mice modulated MDSC and macrophage subsets in the BM. Flow cytometric analysis and representative quantification of (A, B) MDSC subsets, G-MDSC (CD11b+Ly6C+/Ly6G+) and M-MDSC (CD11b+Ly6G Ly6C+) from the BM of CTRL, KC—HPC and KC-HPC-API treated mice. Data are presented as the mean±S.D. of CTRL (n=3-4), KC-HPC (n=4) and KC-HPC-API (n=3-4) mice. *p<0.05; **p<0.01; ***p<0.001 (by two-tailed t test).

FIG. 10C-D are a series of images depicting API treatment of KC-HPC mice modulated MDSC and macrophage subsets in the BM. Flow cytometric analysis and representative quantification of (C, D) macrophage subsets, M1 (CD11b+Ly6C+/Ly6G− F4/80+CD206 MHCII+) and M2 (CD11b+Ly6C+/Ly6G− F4/80+CD206+MHCII−), from the BM of CTRL, KC—HPC and KC-HPC-API treated mice. Data are presented as the mean±S.D. of CTRL (n=3-4), KC-HPC (n=4) and KC-HPC-API (n=3-4) micc. *p<0.05; **p<0.01; ***p<0.001 (by two-tailed t test).

FIG. 11A-D are a series of graphs depicting API treatment of KC-HPC modulated the cell numbers of MDSC and Macrophage subsets in the bone marrow. (A. and B.) Absolute cell numbers of MDSC subsets, G-MDSC (CD11b+Ly6C+/−Ly6G+) and M-MDSC (CD11b+Ly6G-Ly6C+), and (C. and D.) macrophage subsets, M1 (CD11b+Ly6C+Ly6G-F4/80+CD206-MHCII+) and M2 (CD11b+Ly6C+Ly6G-F4/80+CD206+MHCII−), from the BM of CTRL, KC—HPC and KC-HPC-API treated mice. Data are presented as the mean±CTRL (n=3-4), KC-HPC (n=3-4) and KC-HPC-API (n=3-4) mice. *p<0.05; **p<0.01 (by two-tailed t test).

FIG. 12A-C are a series of images depicting API treatment of KC-HPC mice decreased tumor burden. Tumor growth curves representing (A) tumor volume and (B) tumor area in KC-HPC mice treated with API. (C) Representative images of KC-HPC and KC-HPC-API tumors at the humane endpoint of the study. Data are presented as the mean±<0.05 (by two-tailed/test).

FIG. 13A-D are a series of images depicting API treatment of KC-HPC mice increased CD40 expression on M1 TAM and iNOS in the tumor. (A, B). Flow cytometric analysis of CD40 expression on M1 TAM (CD11b+Ly6C+/−Ly6G-F4/80+CD206-MHCII+) in the tumors of KC-HPC and KC-HPC-API mice. (C, D) WB analysis and representative quantification of iNOS protein in the tumors from KC—HPC and KC-HPC-API mice. Data are presented as the mean±S.D. of KC-HPC (n=4), KC-HPC-API (n=4) mice. *p<0.05 (by two-tailed t test).

FIG. 14A-E are a series of images depicting API treatment of KC-HPC mice increased CD8+ T cell infiltration into the tumor. (A, B) Flow cytometric analysis and (C) absolute cell number of CD8+ T cells (CD3+CD8+CD4−) and its expression of (D, E) CD40L in the tumors of vehicle-treated KC-HPC mice and API treated KC-HPC mice. Data are presented as the mean±S.D. of KC-HPC (n=4), KC-HPC-API (n=4) mice. *p<0.05; **p<0.01 (by two-tailed/test).

FIG. 15A-B are a series of images depicting API increased the infiltration of CD8+T and MHC-II+ cells into the tumor of KC-HPC mice. (A.) 2D UMAP visualization and clustering of immune cells in the tumor slice of KC-HPC and KC-HPC-API mice. (B.) 2D tSNE plot demonstrating the distribution of CD8+ T cells population from the tumors of KC-HPC mice.

FIG. 16A-B are a series of images depicting PC survival curves generated using RNAseq values (A.) Overall survival and (B.) Disease-specific survival of PC patients expressing high SHIP/low miR-155 or vice versa.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention. The following description is not intended to limit the scope of the present description disclosed herein.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

All numerical designations, such as pH, temperature, time, concentration, and molecular weight, including ranges, are approximations which are varied up or down by increments of 1.0, 0.1, 0.01, or 0.001 as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, the term “comprising” is intended to mean that the products, compositions and methods include the referenced components or steps, but not excluding others.

“Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

“Optional” or “optionally” means that the subsequently described event or circumstance may or may not occur, and that the description includes instances where said event or circumstance occurs and instances where it does not.

As used in the specification and claims, the singular form “a”, “an” and “the” include plural references unless the context clearly dictates otherwise. For example, the term “a vector” includes a plurality of vectors.

As used in this specification and the appended claims, the term “or” is generally employed in its sense including “and/or” unless the context clearly dictates otherwise.

As used herein, “about” means approximately or nearly and in the context of a numerical value or range set forth means±15% of the numerical.

As used herein “patient” is used to describe an animal, preferably a human, to whom treatment is administered, including prophylactic treatment with the compositions of the present invention.

As used herein “animal” means a multicellular, eukaryotic organism classified in the kingdom Animalia or Metazoa. The term includes, but is not limited to, mammals. Non-limiting examples include humans, rodents, mammals, aquatic mammals, domestic animals such as dogs and cats, farm animals such as sheep, pigs, cows and horses. Wherein the terms “animal” or the plural “animals” are used, it is contemplated that it also applies to any animals.

The terms “risk or susceptibility” as used herein refers to the determination as to whether a subject would or would not respond to a particular therapy or would or would not develop a particular disease or symptom.

The term “normal” as used herein refers to a sample or patient which are assessed as not having cancer.

“Sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof.

“Tissue sample” or “cell sample” means a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. For instance, a “tumor sample” is a tissue sample obtained from a tumor or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

The term “cell” or “cells” is used synonymously herein and refers to in vitro cultures of mammalian cells grown and maintained as known in the art, as well as biological samples obtained from tumor specimens or normal specimens in vivo.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, the reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

The term “biomarker” is used herein to refer to a molecule whose level of nucleic acid or protein product has a quantitatively differential concentration or level with respect to an aspect of a biological state of a subject. “Biomarker” is used interchangeably with “marker” herein. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a pre-propeptide, a propeptide, a mature peptide or a secreted peptide of the biomarker may be measured. A biomarker can be used either solely or in conjunction with one or more other identified biomarkers so as to allow correlation to the biological state of interest as defined herein. In some embodiments, the expression of mRNA155 is used as a biomarker.

The term “gene expression product” or “expression product” as used herein refers to an RNA transcribed from a gene (either pre- or post-processing) or an amino acid (e.g. a polypeptide, protein, or peptide regardless of any secondary modifications, such as glycosylation, lipidation or phosphorylation) encoded by the gene and generated by the gene when the gene is transcribed (either pre- or post-modification) and translated. An agent is said to increase gene expression if the application of a therapeutically effective amount of the agent to a cell or subject results in an increase in either an RNA or polypeptide expression product or both. An agent is said to decrease gene expression if the application of a therapeutically effective amount of the agent to a cell or subject results in a decrease in either an RNA or polypeptide expression product or both.

The term “expression level” as used herein refers to detecting the amount or level of expression of a biomarker of the present invention. The act of actually detecting the expression level of a biomarker refers to the act of actively determining whether a biomarker is expressed in a sample or not. This act can include determining whether the biomarker expression is upregulated, downregulated or substantially unchanged as compared to a control level expressed in a sample. The expression level in some cases may refer to detecting transcription of the gene encoding a biomarker protein and/or to detecting translation of the biomarker protein.

The term “differential expression” as used herein refers to qualitative or quantitative differences in the temporal and/or spatial gene expression patterns within and among cells and tissues. A differentially expressed gene may qualitatively have its expression altered, including an activation or inactivation, such as in normal versus diseased tissue. Genes may be turned off or on in a given state relative to another state thus allowing comparison of two or more states. A qualitatively regulated gene may exhibit an expression pattern within a state or cell type that can be detectable by standard techniques. Alternatively, the difference in expression may be quantitative such that expression of the gene is modulated, up-regulated (resulting in an increased amount of transcript), or downregulated (resulting in a decreased amount of transcript). The degree to which expression varies needs to be large enough to quantify via standard characterization techniques such as expression arrays, quantitative reverse transcriptase PCR, Northern blot analysis, real-time PCR, in situ hybridization, and RNase protection.

The term “expression profile” as used herein refers to a genomic expression profile, for example an expression profile of microRNAs or proteins. The profiles may be generated by any means for determining a level of a nucleic acid sequence, e.g. quantitative hybridization of microRNA, labeled microRNA, amplified microRNA, cDNA, quantitative PCR, ELISA for quantitation, etc. For proteins, the profiles may be generated by any means for determining a level of a protein, e.g. Western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF), mass spectrometry, microcytometry, microarray, microscopy, fluorescence activated cell sorting (FACS), flow cytometry, and assays based on a property of the protein including but not limited to DNA binding, ligand binding, or interaction with other protein partners. The profile must allow for the analysis of differential gene expression between two samples.

The term “baseline level” or “control level” of biomarker expression or activity refers to the level against which biomarker expression in the test sample can be compared. In some embodiments, the baseline level can be a normal level, meaning the level in a sample from a normal patient. This allows a determination based on the baseline level of biomarker expression or biological activity, whether a sample to be evaluated for disease cell growth has a measurable increase, decrease, or substantially no change in biomarker expression as compared to the baseline level. The term “negative control” used in reference to a baseline level of biomarker expression generally refers to a baseline level established in a sample from the subject or from a population of individuals which is believed to be normal (e.g. non-tumorous, not undergoing neoplastic transformation, not exhibiting inappropriate cell growth). In other embodiments, the baseline level can be indicative of a positive diagnosis of disease (e.g. positive control). The term “positive control” as used herein refers to a level of biomarker expression or biological activity established in a sample from a subject, from another individual, or from a population of individuals, where the sample was believed, based on data from that sample, to have the disease (e.g. tumorous, cancerous, exhibiting inappropriate cell growth). In other embodiments, the baseline level can be established from a previous sample from the subject being tested, so that the disease progression or regression of the subject can be monitored over time and/or the efficacy of treatment can be evaluated.

The terms “overexpression” and “underexpression” as used herein refers to the expression of a gene of a patient at a greater or lesser level, respectively, than the normal or control expression of the gene, as measured by gene expression product expression such as mRNA or protein expression, in a sample that is greater than the standard of error of the assay used to assess the expression.

Expression of genes/transcripts and/or polypeptides encoded by the genes represented by the biomarkers of the present invention can be measured by any of a variety of methods known in the art. In general, expression of a nucleic acid molecule (e.g. RNA or DNA) can be detected by any suitable method or technique of measuring or detecting gene or polynucleotide sequence or expression. Such methods include, but are not limited to, polymerase chain reaction (PCR), reverse transcriptase PCR (RT-PCR), in situ PCR, quantitative PCR (q-PCR), in situ hybridization, Southern blot, Northern blot, sequence analysis, microarray analysis, detection of a reporter gene, or any other DNA/RNA hybridization platforms.

The term “quantifying” or “quantitating” when used in the context of quantifying transcription levels of a gene can refer to absolute or relative quantification. Absolute quantification can be achieved by including known concentration(s) of one or more target nucleic acids and referencing the hybridization intensity of unknowns with the known target nucleic acids (e.g. through the generation of a standard curve). Alternatively, relative quantification can be achieved by comparison of hybridization signals between two or more genes, or between two or more treatments to quantify the changes in hybridization intensity and, by implication transcription level.

A therapeutic agent is an atom, molecule, or compound that is useful in the treatment of a disease to induce a desired pharmacological and/or physiological effect on a subject when administered in a therapeutically effective amount. Examples of therapeutic agents include, but are not limited to, antibodies, antibody fragments, immunoconjugates, drugs, cytotoxic agents, pro-apoptotic agents, toxins, nucleases (including DNAses and RNAses), hormones, immunomodulators, chelators, boron compounds, photoactive agents or dyes, radionuclides, oligonucleotides, interference RNA, siRNA, RNAi, anti-angiogenic agents, chemotherapeutic agents, cytokines, chemokines, prodrugs, enzymes, binding proteins or peptides or combinations thereof.

“Administration” or “administering” is used to describe the process in which compounds of the present invention, alone or in combination with other compounds, are delivered to a patient. The composition may be administered in various ways including, but not limited to, oral; parenteral; intrathecal; intramuscular; subcutaneous; etc. Each of these conditions may be readily treated using other administration routes of compounds of the present invention to treat a disease such as cancer.

“Parenteral administration” as used herein refers to modes of administration other than enteral and topical administration, usually by injection, and includes, but is not limited to, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

“Treatment” or “treating” as used herein refers to any of: the alleviation, amelioration, elimination and/or stabilization of a symptom, as well as delay in progression of a symptom of a particular disorder. For example, “treatment” of cancer may include any one or more of the following: amelioration and/or elimination of one or more symptoms associated with the cancer, reduction of one or more symptoms of the cancer, stabilization of symptoms of the cancer, and delay in progression of one or more symptoms of the cancer.

“Prevention” or “preventing” as used herein refers to any of: halting the effects of the cancer, reducing the effects of the cancer, reducing the incidence of the cancer, reducing the development of the cancer, delaying the onset of symptoms of the cancer, increasing the time to onset of symptoms of the cancer, and reducing the risk of development of the cancer.

The term “prognosis” refers to the determination or prediction of the course of disease or condition or to monitoring disease progression or regression from one biological state to another. Prognosis can include the determination of the time course of a disease, with or without treatment. Where treatment is included, the prognosis includes determining the efficacy of the treatment for the disease or condition.

The term “biological state” as used herein refers to the result of the occurrence of a series of biological processes. As the biological processes change relative to each other, the biological state also changes. One measurement of a biological state is the level of activity of biological variables such as biomarkers, parameters, and/or processes at a specified time or under specified experimental or environmental conditions. A biological state can include, for example, the state of an individual cell, a tissue, an organ, and/or a multicellular organism. A biological state can be measured in samples taken from a normal subject or a diseased subject thus measuring the biological state at different time intervals may indicate the progression of a disease in a subject. The biological state may include a state that is indicative of disease (e.g. diagnosis); a state that is indicative of the progression or regression of the disease (e.g. prognosis); a state that is indicative of the susceptibility (risk) of a subject to therapy for the disease; and a state that is indicative of the efficacy of a treatment of the disease.

The terms “favorable outcome” or “favorable prognosis” or “good prognosis” as used herein refers to long time to progression, long term survival, and/or good response. Conversely, an “unfavorable outcome” or “unfavorable prognosis” or “poor prognosis” refers to short time to progression, short term survival, and/or poor response.

A cancer is “responsive” to a therapeutic agent or there is a “good response” to a treatment if its rate of growth is inhibited as a result of contact with the therapeutic agent, compared to its growth in the absence of contact with the therapeutic agent. Growth of a cancer can be measured in a variety of ways, for instance, the characteristic, e.g., size of a tumor or the expression of tumor markers appropriate for that tumor type may be measured.

A cancer is “non-responsive” or has a “poor response” to a therapeutic agent or there is a poor response to a treatment if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or the expression of tumor markers appropriate for that tumor type may be measured.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W Easton Pennsylvania, Mack Publishing Company, 19^{th} ed.) describes formulations which can be used in connection with the subject invention.

For case of administration, the subject compounds may be formulated into various pharmaceutical forms. As appropriate compositions there may be cited all compositions usually employed for systemically or topically administering drugs. To prepare the pharmaceutical compositions of this invention, the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for administration orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their case in administration, tablets and capsules often represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g. as a transdermal patch, as a spot-on or as an ointment.

As used herein, the term “therapeutically effective amount” refers to that amount of a therapy (e.g., a therapeutic agent or vector) sufficient to result in the amelioration of cancer or one or more symptoms thereof, prevent advancement of cancer, or cause regression of cancer. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. The compositions used in the present invention may be administered individually, or in combination with or concurrently with one or more other therapeutics for cancer.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequently. Administration can also be continuous and adjusted to maintaining a level of the compound within any desired and specified range. In some embodiments, the dosage is at least 3 times per week.

The administration of the composition can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The term “pancreas associated diseases” or “pancreatic disease” as used herein refers to diseases which affect functioning of the pancreas. Examples include, but are not limited to, pancreatitis such as acute, chronic, and autoimmune pancreatitis; pancreatic tumors such as primary epithelial and mesenchymal tumors, lymphomas, and secondary tumors; pancreatic cancer such as pancreatic ductal adenocarcinoma; cystic neoplasms, such as serous cystadenoma, mucinous cystic neoplasm, and intraductal papillary mucinous neoplasm; and cystic fibrosis.

The term “cancer”, “tumor”, “cancerous”, and malignant” as used herein, refer to the physiological condition in mammals that is typically characterized by unregulated cell growth. In particular, solid tumor cancers are contemplated for treatment herein. In some embodiments, the cancer to be treated is pancreatic cancer, including pancreatic ductal adenocarcinoma. Non-limiting examples of a cancer that can be treated with the intended use described herein include, but are not limited to, the following: pancreatic cancer such as, but not limited to, pancreatic ductal adenocarcinoma, insulinoma, gastrinoma, glucagonoma, vipoma, somatostatin-secreting tumor, and carcinoid or islet cell tumor; lymphomas such as but not limited to Hodgkin's lymphoma, non-Hodgkin's lymphoma; bone and connective tissue sarcomas; glial brain tumors (i.e., gliomas); breast cancer including but not limited to ductal carcinoma, adenocarcinoma, lobular (cancer cell) carcinoma, intraductal carcinoma, medullary breast cancer, mucinous breast cancer, tubular breast cancer, papillary breast cancer, Paget's disease, and inflammatory breast cancer; adrenal cancer; thyroid cancer; pituitary cancers; eye cancers; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, placancercytoma, verrucous carcinoma, and oat cell (cancer cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor); prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

The following non-limiting examples illustrate exemplary compositions and methods of treatment thereof in accordance with various embodiments of the disclosure. The examples are merely illustrative and are not intended to limit the disclosure in any way.

### Example 1—Apigenin Regulates SHIP-1 Expression by Suppressing mRNA-155

The efficacy of cancer immunotherapy in PC patients remains limited because of the induction of an immunosuppressive TME, which leads to the inactivation of anti-tumor immune responses^{6, 7, 74}. Targeting a TME enriched with immunosuppressive cells, specifically MDSC and TAM, is an important strategy to improve the success of immunotherapy in PC. Pre-clinical studies have elucidated the critical role of MDSC and TAM not only in pancreatic tumor progression and metastasis but also in conferring resistance to chemotherapy^{75, 76}. Furthermore, accumulation of MDSC and pro-tumor TAM in the TME have been shown to correlate with metastatic relapse, leading to reduced survival in PC patients^{77, 78}.

In earlier preclinical studies, the inventors reported that apigenin depleted immunosuppressive MDSC and TAM from the TME, induced SHIP-1 expression, increased tumoricidal macrophages, enhanced anti-tumor immune responses and reduced tumor burden in different PC models¹². The inventors have now found that apigenin depleted miR-155 expression in murine as well as human PC cell lines, which corresponds to the current results regarding increased apoptosis and reduced cell viability. These results support that API treatment of OPC mice induced necrosis of pancreatic tumor cells validated by H&E staining¹². The inhibition of PC cell growth by API may be due to the downregulation of PI3K/AKT and MAPK/MEK/ERK pathways as these kinases are downstream of Kras oncogene which is mutated in 90% of patients with PC^{79, 80}. It is interesting to note that both API and miR-155 have common apoptotic targets in cancer cells such as caspase 3, caspase 9, FAS and Bcl^{51, 81, 82}. API depletes anti-apoptotic Bc12 and increases pro-apoptotic caspase 3 whereas miR-155 increases anti-apoptotic Bcl2 and decreases pro-apoptotic caspase 3 in cancer cells^{51, 82}. In addition, API may inhibit topoisomerase I-catalyzed DNA religation and enhance gap junctional intercellular communication through induction of phosphorylation of the ataxia-telangiectasia mutated (ATM) kinase and histone H2AX, two key regulators of the DNA damage response⁸³.

MIR-155, a prognostic blood-based biomarker linked to poor survival in PC patients, is known to be implicated in the development of pancreatic tumors from pancreatic intraepithelial neoplasia (PanIN) lesions to adenocarcinoma and is overexpressed in PC^{23, 24, 84, 85}. Overexpression of miR-155 promotes gemcitabine chemoresistance, while API has been shown to reduce this chemoresistance in human PC cell lines^{50, 86}. The data further demonstrate that depletion of miR-155 using an miR-155 inhibitor reduced the viability of PC cells, thus supporting the role of miR-155 in PC growth and development. Moreover, combined API and miR-155 inhibitor treatment results in additional depletion of miR-155 which corresponds with synergistic reduction of PC cell viability this supporting the use of both API and miR-155 inhibitor as an adjuvant therapy for PC. Further in vitro studies are being performed regarding API regulation of apoptotic pathways via targeting miR-155 in human and mouse pancreatic adenocarcinoma cells.

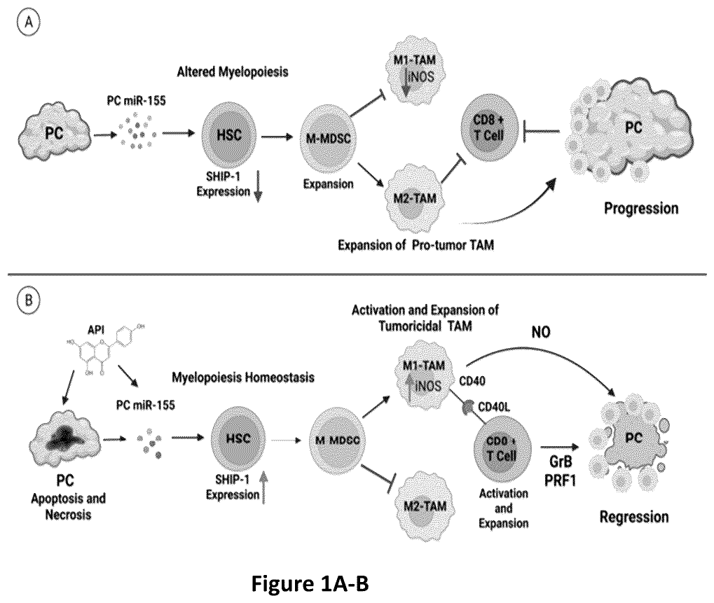

The previous in vivo studies have shown that apigenin treatment in experimental models of PC induces anti-tumor immune responses by depleting proinflammatory TDFs, MDSC and protumor M2-TAM as well as upregulating tumoricidal M1 TAM and SHIP-1 expression in the tumors from mice^{12, 55}. Previous studies have shown that tumoricidal macrophage activity via production of iNOS/NO and CD40-CD40L interactions between macrophages and T cells, respectively, as well as induction of T-cell responses have tumoricidal effects⁸⁷. The results show that M1 TAM in KC-HPC-API mice exhibit upregulation of CD40, which corresponded with an increase in their production of iNOS and coincided with tumor regression. In addition, CD40 expressing macrophages interacting with CD40L on CD8+ T cells has been shown to enhance cytotoxic activity⁸⁷⁻⁸⁹. The previous results show that API increases IFN-γ production from intratumoral CD8+ T cells as well as perforin and granzyme B and that this contributed to low tumor burden in PC-bearing mice¹². These are the proposed mechanisms responsible for the tumor reduction induced by API in models with PC (FIG. 1).

Overexpression of miR-155 has also been reported in both G-MDSC and M-MDSC derived from mice with lung cancer⁹⁰. Dexamethasone has been shown to promote the expansion of MDSC and upregulate miR-155 expression in BM cells, with its effects abolished by depleting miR-15590. The inventors are the first to show herein that apigenin treatment targets and suppresses miR-155 gene expression in the BM and pancreatic tumor, thereby elevating the SHIP-1 gene and protein expressions that corresponded with an increase in tumoricidal M1 TAM percentages and the infiltration of effector cytotoxic CD8+ T cells in tumors from mice (FIG. 1). This correlated with a reduction in proportions of pro-tumor M2-TAM, G-MDSC and M2 macrophages in the BM, thereby increasing anti-tumor immune responses in mouse models of PC. It has been proposed that G-MDSC may develop into tumor associated neutrophils (TAN) in the TME91. These TAN can be tumoricidal N1 or protumor N2 similar to the M1 and M2 TAM nomenclature⁹¹. More recently, N1 TAN have been identified as a new target for the treatment of PC^{91, 92}. The inventors are currently investigating G-MDSC development into TAN in the TME of pre-clinical PC models.

Downregulation of SHIP-1 expression is implicated in chronic myeloid leukemia, Crohn's Disease, T cell leukemia, SLE, ulcerative colitis and Systemic Lupus Erythematous, both in humans and mice^{26, 31}, as well as in preclinical models of PC^{11, 12, 35} SHIP-1 expression is regulated in immune and myeloid cells, including macrophages, by cytokine and chemokine signaling^{30, 33} and is one of the targets of miR-155^{28, 30}, impacting tumor immunity. It is also implicated in the regulation of macrophage polarization in SHIP-1 knockout mice, where they exhibit an immunosuppressive M2 macrophage (pro-tumor) phenotype⁹³. The inventors have also reported the expansion of immunosuppressive M2 macrophage in SHIP-1-deficient mice²⁹. Therefore, SHIP-1 acts as a tumor suppressor, preventing metastasis in pre-clinical cancer models⁹⁴.

Here, the inventors are the first to show greater miR-155 expression in the BM and tumors of SHIP^{KO}-HPC mice compared with SHIP^{WT}-HPC mice indicating the role of SHIP-1 as tumor suppressor and miR-155 as an oncogene. This is interesting because it suggests that SHIP-1 may be acting in a negative feedback loop to repress miR-155 expression. The inventors have reported that SHIP^{KO}-HPC mice have a significant increase in M2-like TAM and a significant decrease in M1-like TAM in the tumor compared with SHIP^{WT}-HPC mice¹². The results support the notion that enhanced SHIP-1 expression reduces immunosuppressive TAM and that this promotes anti-tumor immune responses in the pancreatic TME. Therefore, amplification of SHIP-1 expression through suppression of miR-155 by API or miR-155 inhibitor or both can be a novel means to enhance the anti-tumor immune responses in the pancreatic TME. The inventors are currently performing studies to identify other SHIP-1 regulators such as miR-210 and post-translational modification events such as proteasome degradation^{26, 95-97} that could skew M-MDSC and the balance of M1 vs. M2 TAM in the tumors from PC mice.

miR-155 regulates a plethora of biological properties which include Toll-like receptor (TLR) activation on monocytes and macrophages that facilitate pro-inflammatory cellular responses⁹⁸. The inventors are currently exploring changes in TLR expression on TAM subsets from the API and vehicle-treated PC models. In addition, miR-155 directly targets and transcriptionally suppresses Suppressor of Cytokine Signaling 1 (SOCS1) that influence the development of immunosuppressive regulatory T cells (Treg) and Th17 cells⁹⁹. The inventors reported a significant reduction in intratumoral and splenic Treg percentages from the PC mouse model treated with API12. Wang et al., have shown that the overexpression of miR-155 coincides with an increase in Th17/Treg percentages in the blood of patients with acute pancreatitis⁹⁹. Huang et al., reported that miR-155 enhanced pancreatic cancer cell invasiveness by modulating the STAT3 signaling pathway through SOCS1¹⁰⁰. Therefore, miR-155 regulation of SHIP-1 and SOCS1 expressions can be potential biomarkers and therapeutic targets for the treatment of pancreas-associated diseases (i.e., pancreatitis and pancreatic cancer).

miR-155 targets and binds to the 3′-UTR regions of SHIP-1 transcript (which is a highly conserved binding site), in vitro, via luciferase reporter assay^{28, 101}. In addition, O'Connell R. M. et al. have reported that retroviral expression of miR-155, in vivo, targets SHIP-1 which alters the hematopoietic compartment causing a phenotype similar to myeloproliferative disorder (MPD)²⁸. These authors have also demonstrated a similar MPD phenotype when silencing SHIP-1 using siRNA against SHIP-1, in vivo²⁸. The inventors have also reported similar findings of MPD in transgenic SHIP-KO mice and mouse models of PC^{11, 12, 29}. In addition, the inventors have reported that miR-155 expressing PC cells co-cultured with control splenocytes suppressed SHIP-1 gene and protein expression, in vitro¹¹. The inventors use pharmacologic and genetic tools to mechanistically show that PC-induced miR-155 targets and downregulates SHIP-1 expression using in vitro and in vivo model system.

The association between dietary flavonoids, including apigenin, and their role in cancer was investigated in patients with ovarian cancer¹⁰², breast cancer¹⁰³, and lung cancer¹⁰⁴. The data show an inverse association between the intake of flavonoids, including apigenin, and incidence of many types of solid cancers. In a prospective cohort study of patients with resected colon cancer, those treated with a flavonoid mixture (apigenin and EGCG) had a reduced rate of recurrence of colon neoplasia¹⁰⁵. One open-label clinical study tested a combination therapy including apigenin, ferulic acid, gamma oryzanol, and silymarin in patients with Alzheimer's, Parkinson's and multiple sclerosis¹⁰⁶. The data show an improvement in the pathology of neurodegenerative disease.

An ongoing clinical trial has been testing an apigenin-rich celery-banana bread in high-risk breast cancer patients (Clinical Trials Identifier NCT03139227). A phase I clinical trial of LNA-modified anti-miR-155 (MRG-106) has also been initiated (Clinical Trials Identifier NCT02580552). This trial has been evaluating patients diagnosed with cutaneous T-cell lymphoma (CTCL) of the mycosis fungoides subtype, and the results are encouraging. Preliminary data demonstrate that intra-tumoral injection of MRG-106 results in improved cutaneous lesions with almost no side effects. In 2018, a phase II clinical trial was also initiated to further evaluate the efficacy of MRG-106 against CTCL (Clinical Trials Identifier NCT03713320). The preclinical data using apigenin and miR-155 inhibitor as antineoplastic agents strongly suggest the clinical utility of these agents as adjuvants in PC patients.

### Results

API Suppresses miR-155 Gene Expression and Inhibits the Viability of Pancreatic

### Cancer Cells In Vitro

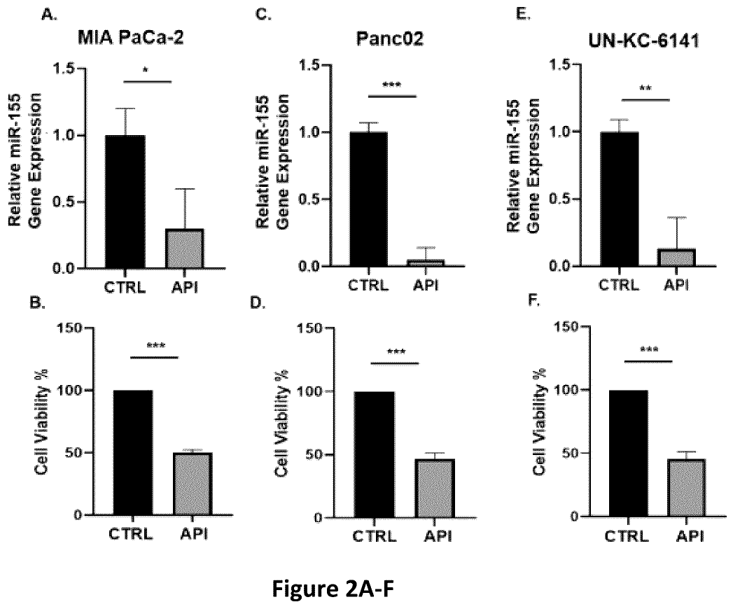

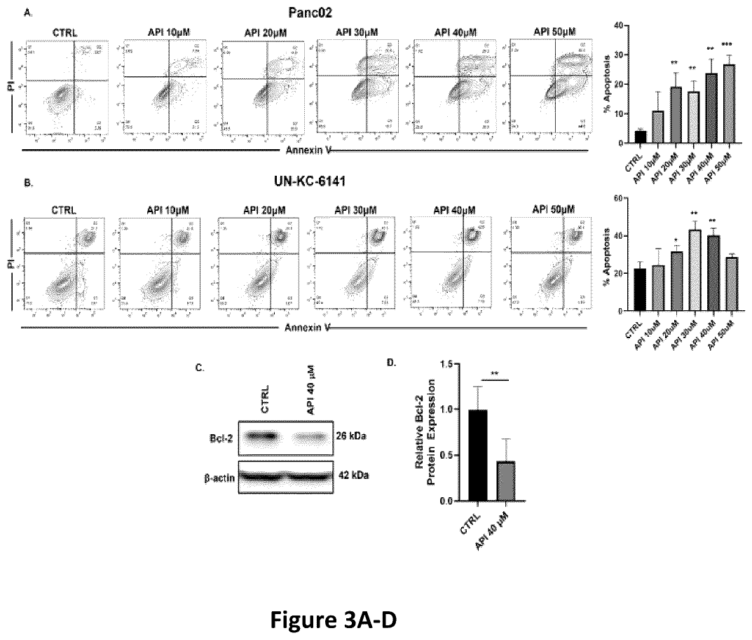

MiR-155 is known to be overexpressed in different cancers, including PC^{21, 23}. The inventors first assessed whether apigenin could modulate the expression of miR-155 in PC cells. Murine PC cell lines Panc02 and UN-KC-6141 as well as the human PC cell line MiaPaCa-2 were treated with API (40 μM) or DMSO vehicle for 24 h and the expression of miR-155 was examined using RT-qPCR assay. It was found that API significantly suppressed miR-155 gene expression in three PC cell lines (FIG. 2). Since API is known to inhibit the growth of cancer cells, including PC cells^{39-43, 47-50}, the same PC cell lines were treated with API (40 μM) or DMSO (vehicle) for 24 h and examined cell viability using MTT assay. The data show that API significantly inhibited the viability of PC cell lines (FIG. 2), indicating the anti-carcinogenic effect of API in PC. Moreover, Panc02 and UN-KC-6141 cells were then treated with API at a dose dependent concentration (10, 20, 30, 40, 50 μM) and detected apoptosis using flow cytometry (Annexin V and PI). The results show that API induces apoptosis in both Panc02 and UN-KC-6141 cells with increasing concentration of API (FIG. 3A, B). Bcl-2 protein expression in UN-KC-6141 cells treated with API (40 μM) was then assessed and the WB results show a significant decrease in Bcl-2 protein in API treated PC cells (FIG. 3C).

Inhibition of miR-155 Decreases the Viability of PC Cells In Vitro

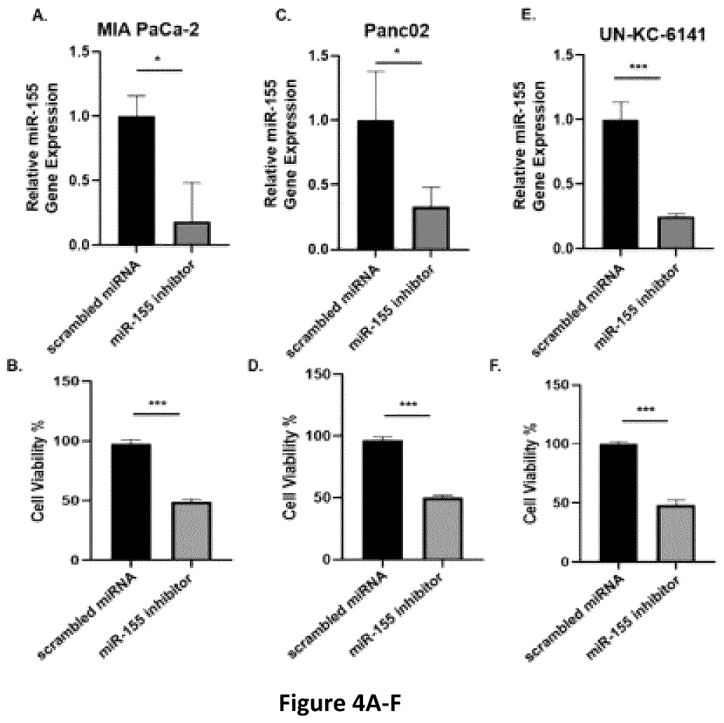

miR-155 is implicated in the development of pancreatic tumors from pancreatic intraepithelial neoplasia (PanIN) lesions, and is overexpressed in PC²³. The inventors examined whether the depletion of miR-155 modulated PC viability. Murine and human PC cell lines were transfected with miR-155 inhibitor (i.e., MMU-MIR-155-5P/HSA-MIR-155-5P) and scrambled miRNA at a concentration of 100 nM for 24 h and cell viability determined using an MTT assay. The expression of miR-155 was examined using an RT-qPCR assay as well. The results show that miRNA-155 inhibitor significantly depleted miR-155 gene expression, and correspondingly, suppressed the viability of three different PC cell lines compared to scrambled miRNA (FIG. 4A-F), indicating the vital role that miR-155 plays in the growth of PC.

Concomitant API and miR-155 Inhibitor Treatment Synergistically Suppresses miR-155 and the Viability of PC Cells In Vitro

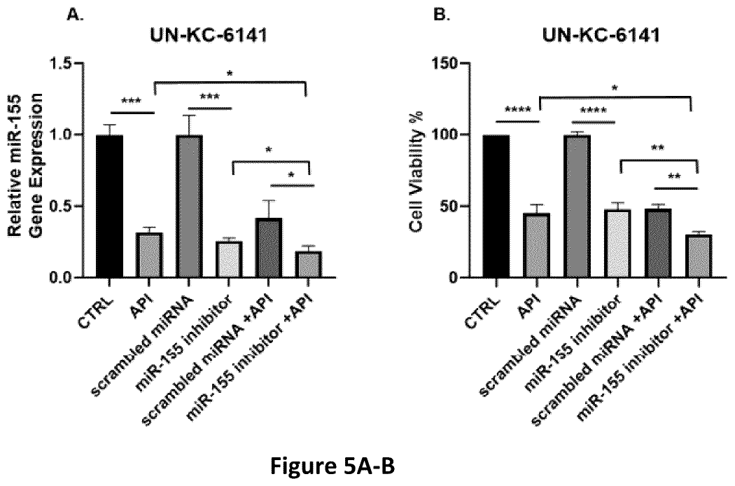

Since miR-155 is overexpressed and implicated in the development of PC²³, the inventors then examined whether combining an miR-155 inhibitor with API further inhibits PC cell viability. To test this, the murine PC cell line, UN-KC-6141, was transfected with either miR-155 inhibitor or scrambled miRNA and treated with API and cell viability and miR-155 gene expression were examined. Interestingly, the combination of API and miR-155 inhibitor synergistically depleted miR-155 gene expression in PC cells and suppressed PC cell viability (FIG. 5A, B), suggesting the anti-cancer activity of both API and miR-155 inhibitor against PC.

API Decreases the Production of miR-155 and this Correlates with an Increase in SHIP-1 Expression in HPC, OPC and KC-HPC Mouse Models

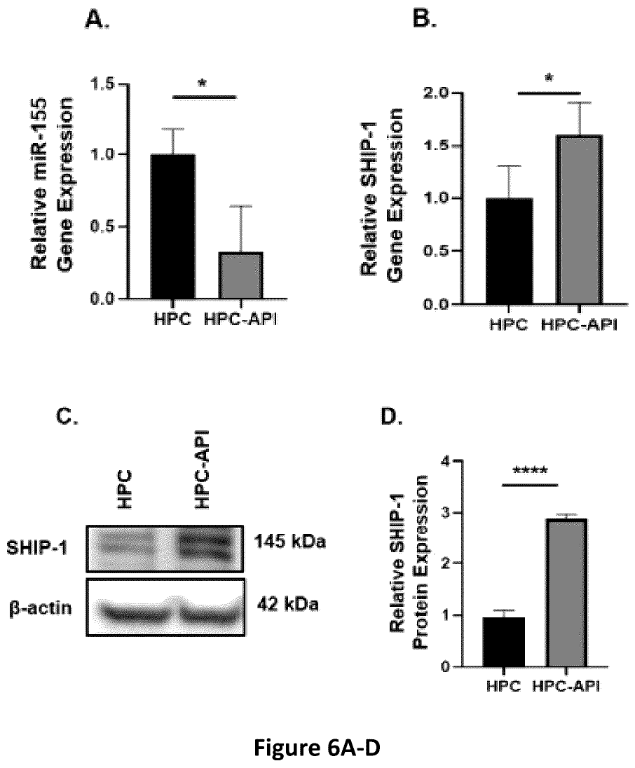

Earlier studies have shown the anti-tumor activity of API in pre-clinical experimental models of cancer^{41, 62-68}. The inventors have reported that API increased survival and reduced tumor growth in a heterotopic (H) mouse model of PC (HPC)⁵⁵. To investigate if API targets miR-155 and regulates SHIP-1 in the TME of an HPC mouse model, HPC mice were treated with API at a dose of 25 mg/kg (IP) 3 times per week for 3-4 weeks. The results show that API treatment significantly reduced miR-155 gene expression which, in turn, increased SHIP-1 gene and protein expressions in the tumor of HPC mice compared with vehicle treated HPC mice (FIG. 6A-D).

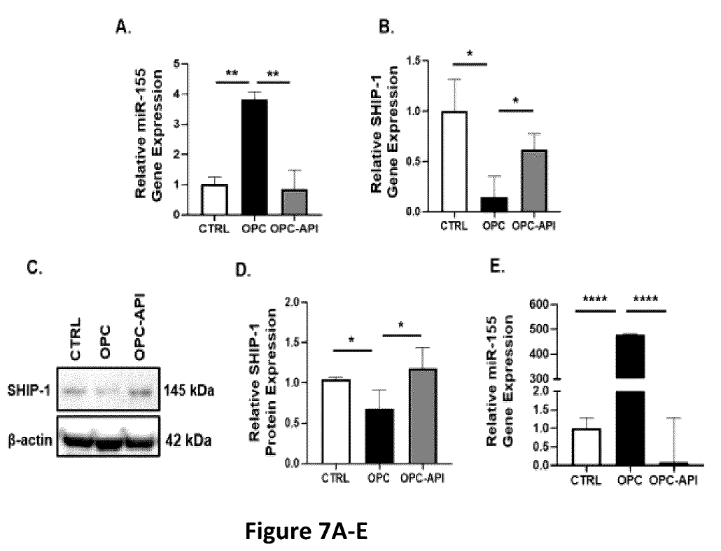

The inventors reported recently that API reduced tumor growth in an orthotopic (O) mouse model of PC (OPC)¹², which is a more clinically relevant model⁶⁹. To investigate if API targets miR-155 and regulates SHIP-1 expression in the BM and tumor of an OPC mouse model, OPC mice were treated with API at a dose of 25 mg/kg (IP) 3 times per week for 2-3 weeks. A significant increase in miR-155 gene expression in the BM of OPC vs. CTRL mice was observed, and API treatment of OPC mice was found to inhibit the overexpression of miR-155 (FIG. 7A). In contrast, SHIP-1 gene and protein expression in the BM was significantly increased in API-treated OPC mice compared with OPC mice (FIG. 7B-D). Looking at the pancreatic tumors from OPC mice, there was very marked overexpression of miR-155 compared with the pancreas of CTRL mice, however due to API treatment of OPC mice a significant decrease in miR-55 gene expression was noticed (FIG. 7E). The finding of increased SHIP-1 expression in the BM of API-treated OPC mice suggests that it may be involved in the regulation of myelopoiesis. In addition, these results corroborate the previous findings that SHIP-1 gene and protein expressions is increased in the pancreatic tumor of API-treated OPC-API mice¹².

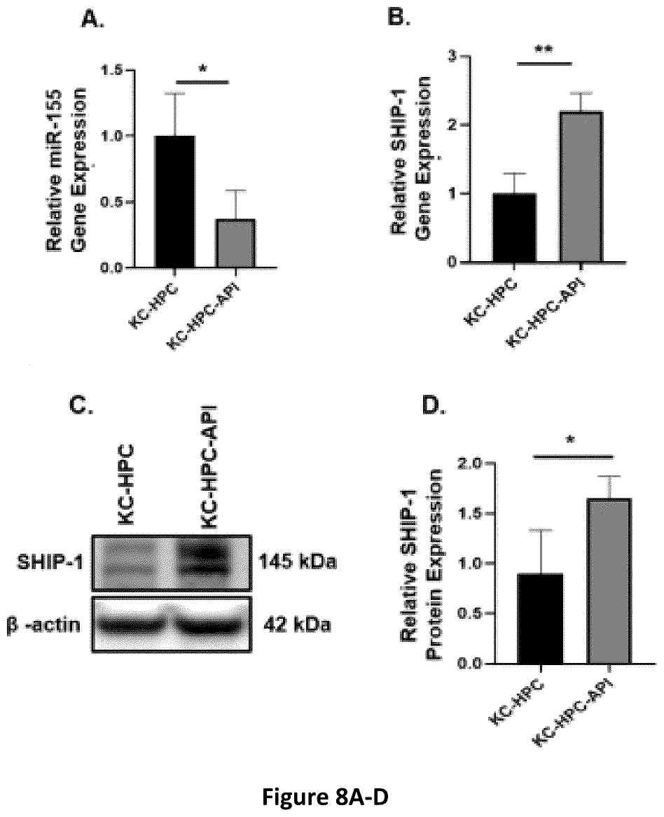

To build on these findings, the inventors then used a novel pancreatic cancer cell line, UN-KC-6141, that resembles human PC due to its expression of a mutated Kras and generated mice harboring these tumor cells, called KC-HPC mice. Such mice were treated with API at a dose of 25 mg/kg (IP) three times per week for 17 days. The inventors examined if API also targets miR-155 and regulates SHIP-1 in the tumors from KC-HPC mouse model. It was found that API treatment significantly reduced miR-155 gene expression in the tumors from KC-HPC mice compared with vehicle treated KC-HPC mice (FIG. 8A), and significantly increased the gene and protein expressions of SHIP-1 (FIGS. 8B-D).

miR-155 Expression in SHIP^{KO}-HPC is Increased Compared to SHIP^{WT}-HPC Mice

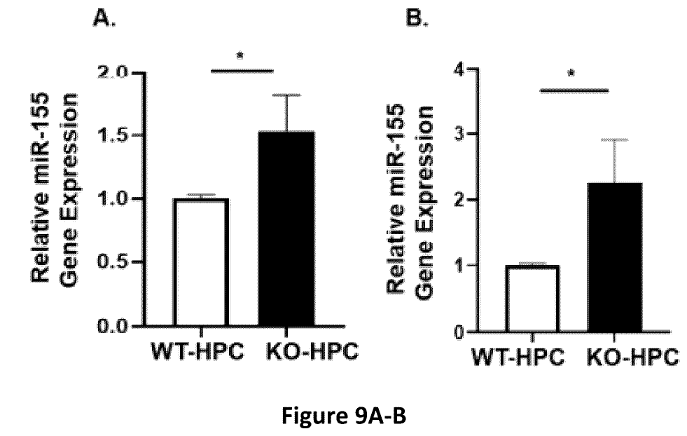

SHIP-1 is known to be involved in the development and function of myeloid cells including MDSC, macrophages and DC30 and is one of the targets of miR-15528.30 impacting tumor immunity. The inventors previously reported downregulation of SHIP-1 expression in immunocompetent C57BL/6 mice with PC¹¹. To investigate the role of miR-155 and SHIP-1 in PC, murine Panc02 cells were heterotopically inoculated into SHIP^{WT} mice and SHIP^{KO} mice, which were designated SHIP^{WT}-HPC and SHIP^{KO}-HPC mice, respectively. Significantly higher miR-155 expression in the BM and tumors from SHIP^{KO}-HPC mice compared with SHIP^{WT}-HPC mice was observed (FIG. 9A, B).

### API Improves Myelopoiesis and Anti-Tumor Responses in KC-HPC Mice

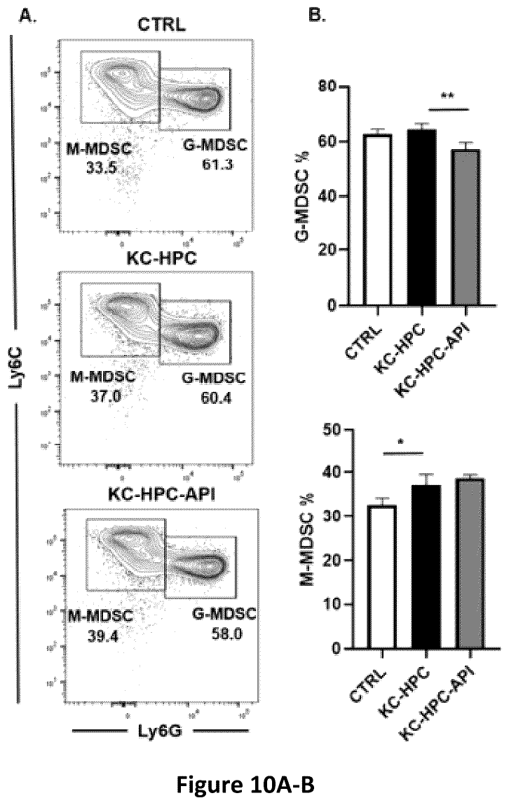

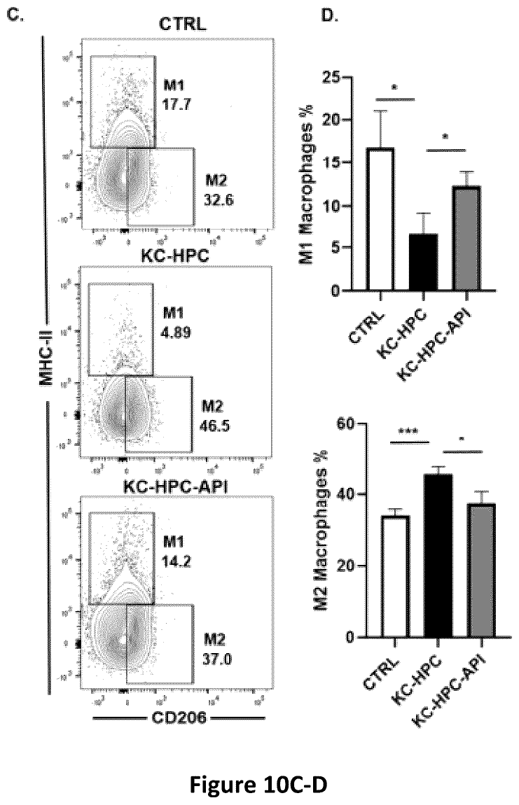

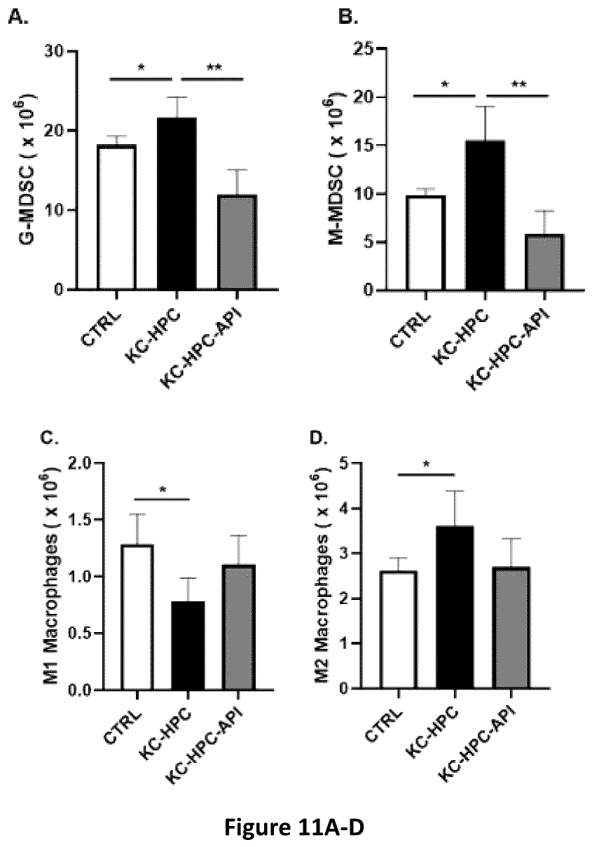

Pancreatic cancer patients are known to have altered myelopoiesis and since API has been shown to target miR-155 in microglia and macrophages^{10, 70-72}, the inventors then examined MDSC and macrophage subsets in the BM of KC-HPC mice. Flow cytometric analysis of BM cells showed that KC-HPC mice had significantly higher percentages of M-MDSCs compared with control mice and API treatment significantly reduced proportions of G-MDSCs without any significant change in proportions of M-MDSCs compared with vehicle treated KC-HPC mice (FIG. 10A, B). The inventors calculated the absolute cell numbers of MDSC subsets in the BM of KC-HPC mice and observed that both G-MDSC and M-MDSC cell numbers were significantly increased and API treatment significantly lowered MDSC subsets absolute cell numbers (FIG. 11A, B). Since macrophages in the BM can be phenotypically characterized as M1 or M2 macrophages, which are tumoricidal and pro-tumor, respectively⁷³, the inventors carried out flow cytometric analysis of the BM of KC-HPC mice and found a significant decrease in M1 and increase in M2 macrophages compared with control mice (FIG. 10C, D). Interestingly, API treatment of KC-HPC mice significantly increased M1 and decreased M2 macrophage percentages in the BM compared with KC-HPC mice (FIG. 10C, D). The inventors also observed that the absolute cell numbers of M1 macrophages were significantly decreased whereas M2 macrophages were significantly increased in BM of KC-HPC mice, however there were no significant changes in M1 and M2 macrophages absolute cell numbers of KC-HPC mice treated with API (FIG. 11C, D).

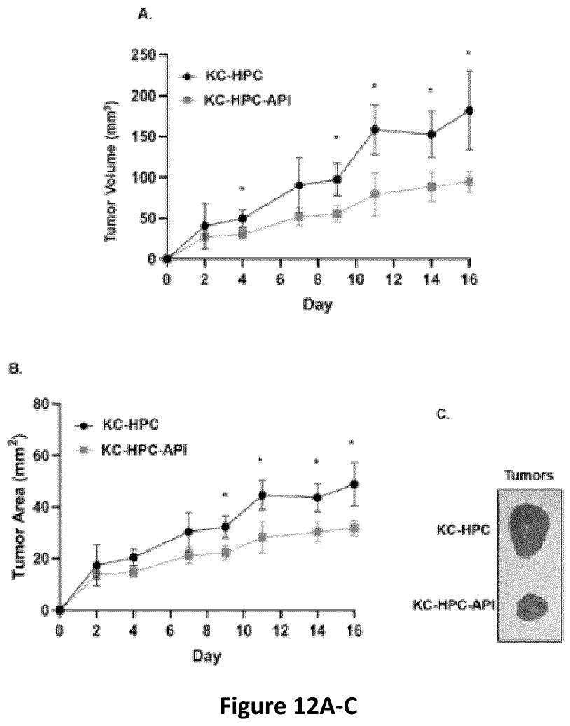

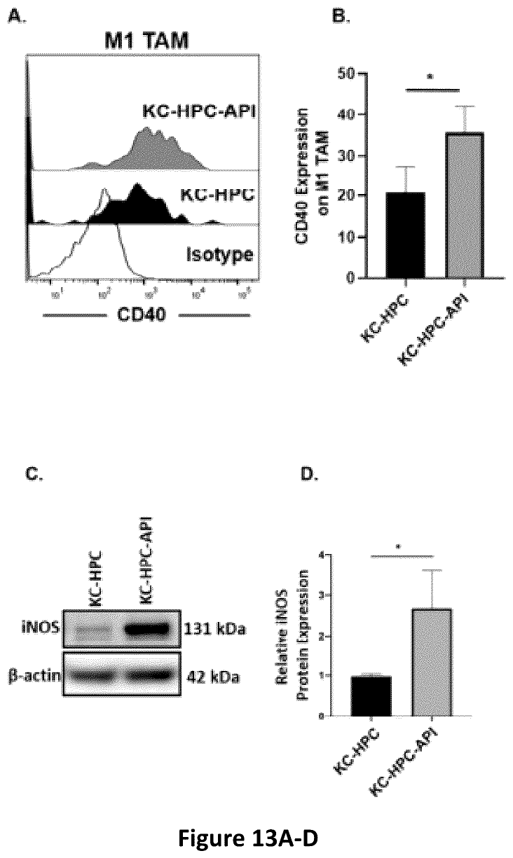

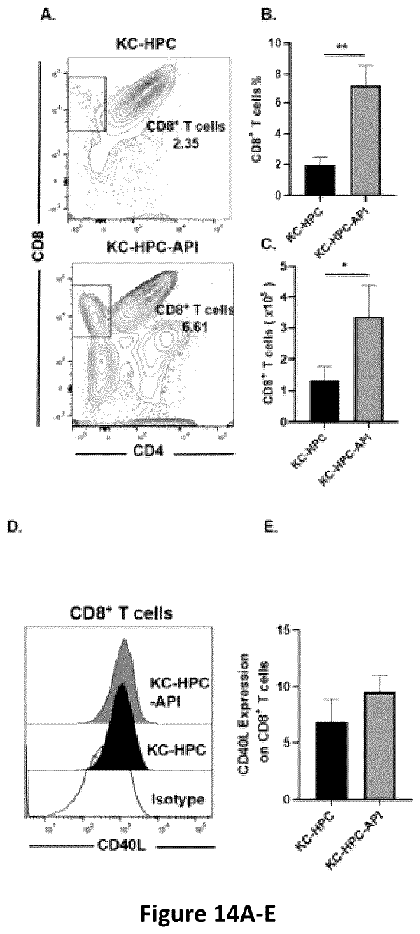

In keeping with the previous report showing that API decreases the tumor weights of PC mice^{12, 55}, the inventors found herein that API slows the growth of tumors in KC-HPC mice, significantly reducing tumor burden (FIG. 12A-C). The inventors then assessed the expression of CD40 (a T cell co-stimulation marker) on M1 TAM (gated as MHC-II⁺ and CD206) within the tumor from KC-HPC vs. KC-HPC-API mice. Flow cytometry showed that CD40 expression was significantly upregulated on M1 TAM and this correlated with a significant increase in expression of iNOS protein in the tumor of KC-HPC-API compared with KC-HPC mice (FIG. 13A-D). Flow cytometric analysis and calculated absolute cell numbers revealed a significant increase in the intra-tumoral CD8⁺ T cells (CD3⁺CD8⁺CD4⁻) infiltrating the tumor of API-treated KC-HPC mice compared with vehicle-treated KC-HPC mice, however there were no significant changes in their expression of CD40L (FIG. 14A-E).

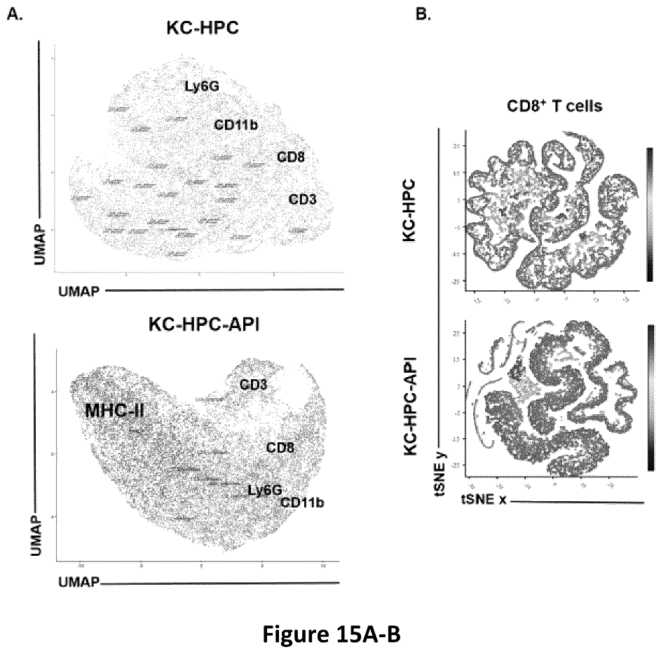

Next, the inventors examined the expression and infiltration of CD8⁺ T cells and MHC-II⁺ cells in the tumor sections of KC-HPC treated with vehicle or API and using AKOYA-CODEX which provides spatial capture of location and phenotype for multiple immune cells in the tumor. Two-dimensional UMAP plots of single cells show that API treated KC-HPC mice demonstrated an increase in MHC-II⁺ cells which was non-detectable in KC-HPC mice (FIG. 15A). The inventors then observed, via 2D tSNE, increased CD8⁺ T cell expression partially located within the tumor plots for the tumors of KC-HPC-API mice compared with vehicle treated KC-HPC mice (FIG. 15B).

Lower Survival is Associated with High miR-155 and Low SHIP-1 Gene Expressions in PC

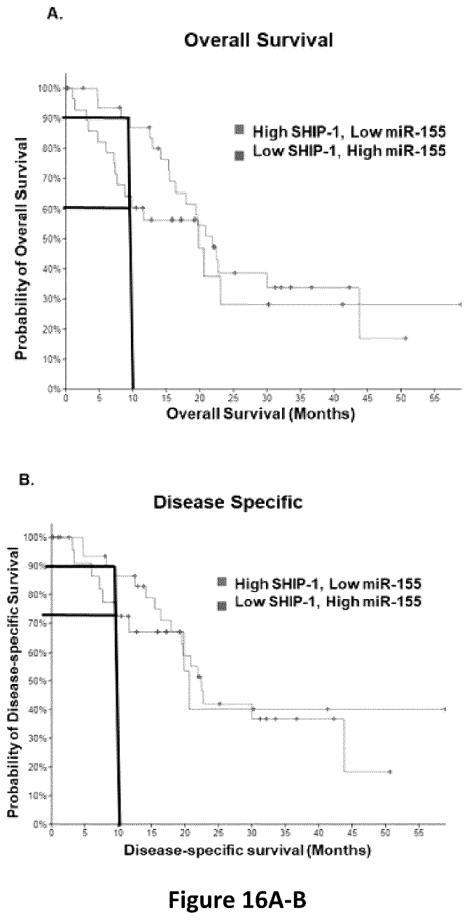

To determine if augmented miR-155 expression in PC patients correlated with reduced SHIP-1 expression, the inventors obtained the RNAseq values for The Cancer Genome Atlas-pancreatic adenocarcinoma database and assembled two groups of case IDs representing (i) high miR155HG expression and low SHIP-1 expression versus (ii) low miR155HG and high SHIP-1 expression (FIG. 16). The overlaps were based on the upper and lower 50th percentiles for both groups. For example, group (i) represents the overlap of the upper 50th percentile of miR155HG expression and the lower 50th percentile of SHIP-1 expression (FIG. 16). While the Kaplan-Meier analysis did not indicate a log-rank p-value representing a distinction between the two sets of case IDs, at the 10-month time period there was a significant difference in survival in PC patients, with lower survival correlating with high miR155HG and low SHIP-1 expression (Table 1).

TABLE 1
Lower survival probability in PC patients assessed at a
single time point for high miR-155HG
and low SHIP-1 expression (FIG. 16).

| Survival Analysis Type | Time Period | Two Proportion Test p-Value |
|---|---|---|
| Overall survival | 10 months | 0.006 |
| Disease specific | 10 months | 0.078 |

### Materials and Methods

### Pancreatic Ductal Adenocarcinoma Cancer Cell Lines

Human PC cell line MiaPaCa-2 was obtained from Mokenge Malafa of H. Lee Moffitt Cancer Center, USA. The murine Panc02 adenocarcinoma cell line originated from C57BL/6 mice [56]. The murine UN-KC-6141 cell line was derived from a C57BL/6 mouse bearing a KrasG12D; Pdx1-Cre (KC) pancreatic tumor⁵⁷. Panc02, MiaPaCa-2 and UN-KC-6141 cell lines were maintained in RPMI 1640 or DMEM (+4.5 g/L D-Glucose, L-Glutamine), supplemented with 10% fetal bovine serum (FBS) (HyClone), 100 U/mL penicillin and 100 μg/mL streptomycin (Gibco, Waltham, MA, USA) at 37° C. in 5% CO₂ incubator. Cultured cells were negative for mycoplasma and viral contamination.

### Bioflavonoid

Apigenin (API) (40,5,7-Trihydroxyflavone, 5,7-Dihydroxy-2-(4-hydroxyphenyl)-4-benzopyrone) (Sigma-Aldrich, St. Louis, MO, USA) was diluted in DMSO (100 mM), stored at −20° C. and later used for assays described herein.

### Cell Viability and Apoptosis Assays of Pancreatic Cancer Cells

The methyl thiazol tetrazolium (MTT) assay was performed as described previously⁵⁸. Briefly, MiaPaCa-2, Panc02 and UN-KC-6141 cells were seeded in 96-well plates at a density of 3000 cells/well and allowed to attach overnight. PC cells were then treated with API (40 μM) or DMSO (1%) as vehicle control for 24 h at 37° C. in 5% CO₂ incubator. After 24 h, media was replaced with 20 μL of MTT (1 mg/mL) (Sigma-Aldrich) and incubated for 3 h at 37° C. in 5% CO₂. MTT was replaced with DMSO, and absorbance was read at 570 nm.

Panc02 and UN-KC-6141 cells were seeded in 6-well plates allowed to grow to 50-60% confluency. Cells were then treated with API at a dose-dependent concentration (10, 20, 30, 40, 50 μM) or DMSO (1%) as vehicle control and incubated for 24 h at 37° C. in a humidified atmosphere of 5% CO₂. Cells were collected and stained with a FITC Annexin V Apoptosis Detection Kit I (BD Bioscience, San Jose, CA, USA) according to the manufacturer's instructions. Acquisition of samples was performed using a flow cytometer BD LSRII (BD Biosciences Immunocytometry Systems, San Jose, CA, USA). FlowJo v10.8 software (TreeStar Inc., Ashland, OR, USA) was used for data analysis.

Transfection of miR-155 Inhibitor into Pancreatic Cancer Cells

MiaPaCa-2, Panc02 and UN-KC-6141 cells (2×10⁵) were grown in 2 mL of growth medium in a 6-well tissue culture plate for 18-24 h until 60-80% confluency. The following solutions were prepared: Solution A) For each transfection, diluted 1 μL of miRNA-155 inhibitors (1 μM) (Table 2) or scrambled negative control miRNA (1 μM) (Qiagen, Germantown, MD, USA) into 50 μL Opti-MEM Transfection Medium (Invitrogen, Waltham, MA, USA). Solution B) For each transfection, diluted 3 μL of Hi-Perfect Transfection Reagent into 50 μL Opti-MEM Transfection Medium. Solution A was added directly to Solution B, mixed gently and the mixture incubated for 10-15 min at room temperature. Cells were then washed once with 2 mL of Opti-MEM Transfection Medium and 900 μL of Opti-MEM Transfection Medium was added to each tube containing 100 μL (Solution A+Solution B), mixed gently and the mixture was overlaid onto the washed cells drop wise. The cells were incubated for 24 h at 37° C. in a CO₂ incubator. After 24 h post-transfection with miRNA-155 inhibitors or scrambled negative control miRNA, cohorts of cells were treated with either API (40 μM) or DMSO (1%) as vehicle control for 24 h at 37° C. in 5% CO₂ incubator. Cells were harvested for different biological assays as described within the cell viability and miRNA extraction sections.

TABLE 2
miRNA-155 inhibitors.

| Qiagen miRCURY LNA | |
|---|---|
| miRNA Inhibitors | Sequence 5′-3′ |
| MMU-MIR-155-5P (mouse) | CCCCTATCACAATTACCA |
| (SEQ ID NO: 1) | TT |
| HSA-MIR-155-5P (human) | CCTATCACGATTAGCATT |
| (SEQ ID NO: 2) | |

### Pancreatic Cancer Murine Models

All female C57BL/6 mice (6-8 weeks of age) described in this study were purchased from Envigo (Indianapolis, IN, USA) and were acclimatize for one week in a pathogen-free animal facility (University of South Florida (USF) vivarium) before injections with PC cells. To generate heterotopic pancreatic cancer (HPC) models, mice were subcutaneously injected with 1.5×10⁵ murine Panc02 (HPC mice) or 5×10⁶ murine UN-KC-6141 cells (KC-HPC mice), in sterile 1× phosphate buffer saline (PBS), in the lower ventral abdomen, while control (CTRL) mice received sterile 1×PBS. Once tumors where palpable, API (25 mg/kg, 100 μL volume) treatments (HPC-API and KC-HPC-API), via intraperitoneal (IP) injections, were started while CTRL, HPC and KC-HPC mice received sterile PBS (vehicle) three times per week until the end of the study^{12, 55}. The HPC models were euthanized 21-28 days post-injection, while the KC-HPC models were euthanized 16-17 days post-injection.

To generate orthotopic pancreatic models (OPC), anesthetized (1.5-3% isoflurane) mice were injected in the neck of the pancreas, via laparotomy⁵⁹, with sterile PBS (CTRL) or 1.25×10⁴ Panc02 cells (OPC), in sterile 1×PBS. Once pancreatic tumors were confirmed through ultrasound imaging (Vevo 2100), a cohort of OPC mice received API (25 mg/kg, 100 μL volume) while CTRL and OPC mice received sterile PBS (vehicle), all via IP, three times per week until the end of the study¹². The endpoint of OPC mice was reached at 16-20 days post-surgery.

Male and Female SHIP^{HET} mice (C57BL/6 background) were kindly received from the Hibbs Lab [60]. These mice were bred at the USF vivarium to obtain SHIP^{KO} and SHIP^{WT}, confirmed by genotyping. To generate SHIP^{KO}-HPC and SHIP^{WT}-HPC mice, these mice (4-6 weeks of age) were SC injected with 1.5×10⁵ Panc02 cells as previously described above. The endpoint of the study was reached at 14 days post-injection.

All the PC models along with their CTRL counterparts were humanely euthanized (CO₂ and cervical dislocation), according to the USF Institutional Animal Care and Use Committee (IACUC) guidelines, as described in the previous study¹². Mouse tissues including, tumors and femur/tibia, were harvested and processed for different biological assays as described below.

### Flow Cytometry

The femur and tibia were cut at the pelvic-hip joint and muscle was removed. The femurs and tibias were cut at the ends and flushed with a 30-gauge needle and 10 cc syringe filled with completed RPMI media until bone was white onto a petri dish. Pelleted bone marrow (BM) cells were treated with Red Blood Cell (RBC) lysis buffer (eBioscience, Waltham, MA, USA), according to the manufacturer's instructions, and then filtered through a 70 μM nylon mesh cell strainer in 1×PBS. All pancreatic tumors were manually cut into ˜1-2 mm³ fragments with a sterile razor blade and then enzymatically digested with Collagenase, type IV (Sigma-Aldrich), DNase, type IV (Sigma-Aldrich) and Hyaluronidase, Type V (Sigma-Aldrich) in Hank's Balanced Salt Solution for 1 h, spun down and washed as described in previous studies⁶¹. Digested pancreatic tumor samples were treated with RBC lysis buffer and then filtered through a 70 μM nylon mesh cell strainer in 1×PBS. Bone marrow and digested pancreatic tumor single cell suspensions were counted with a hemocytometer and then resuspended in 3% FBS/PBS. BM and digested pancreatic tumor cells (1×10⁶) were Fc Blocked (anti-mouse CD16/CD32) and then surface stained for 30 min on ice protected from light with fluorescent anti-mouse antibodies including, CD11b-APC, Ly6C-PE/Cy7, Ly6G-PerCP, F4/80-BV650, MHC—II-BV605, CD206-FITC and CD40-PE/Cy5 along with isotype control antibodies (Biolegend, San Diego, CA, USA) to detect MDSC and macrophage subsets. Digested pancreatic tumor cells (1×10⁶) were also Fc Blocked and then surface stained with fluorescent anti-mouse antibodies including CD3-FITC, CD8-PercP/Cy5.5, CD4-APC/Cy7, and CD40L-APC along with isotype control antibodies (Biolegend) to detect CD8⁺ T cells. Subsequently, all samples were fixed with 2% paraformaldehyde for 15 min on ice protected from light. Acquisition of samples was performed using a flow cytometer BD LSRII (BD Biosciences Immunocytometry Systems). Data analysis was performed using FlowJo v10.8 software (TreeStar Inc.).

miRNA Extraction and TaqMan miRNA Assay

miRNA extraction of PC cells (treated with miR-155 inhibitor and/or API), BM and digested pancreatic tumors cells was performed using a mirVana miRNA Isolation Kit (Applied Biosystems, Waltham, MA, USA) as per the manufacturer's instructions. miRNA was reverse transcribed into cDNA using TaqMan miRNA reverse transcription kit (Applied Biosystems) with gene-specific stem-loop RT primers according to the manufacturer's instructions. cDNA was then loaded onto either mmu-miR-155 or ipu-miR-155 TaqMan miRNA assays (Assay IDs: 002571 and 467534_mat) using TaqMan Fast Universal PCR Master mix (Applied Biosystems) and miR-155 detected with an Eppendorf Master cycler real plex 4. The PCR cycling parameters were 95° C. for 10 min followed by 40 cycles of a denaturing step at 95° C. for 15 s and an annealing/extension step at 60° C. for 1 min. All reactions were performed in triplicate. Relative expression of miR-155 was calculated using the comparative 2 MCt method normalized to a mouse endogenous control gene snoRNA202 and human endogenous control gene U6 (Assay IDs: 001232 and 001093).

### RT-Quantitative PCR (RT-qPCR)

Isolation of total RNA from digested pancreatic tumors and BM cells was performed using a RNeasy Mini Kit (Qiagen), according to the manufacturer's instructions. RT-PCR of quantified and normalized total RNA was performed using a High-Capacity cDNA RT Kit with an RNase Inhibitor (Applied Biosystems), according to the manufacturer's instructions. SHIP-1 and GAPDH (housekeeping gene) mRNA levels were evaluated with an Eppendorf Master cycler real plex 4 using iQ SYBR Green Supermix (Bio-Rad, Hercules, CA, USA) along with the following primers from Integrated DNA Technologies: SHIP-1 forward, 5′-CCA GGG CAA GAT GAG GGA GA-3′ (SEQ ID NO: 3), SHIP-1 reverse, 5′-GGA CCT CGG TTG GCA ATG TA-3′ (SEQ ID NO: 4), and GAPDH forward, 5′-TGA TGG CGT GGA CAG TGG TCA TAA-3′ (SEQ ID NO: 5), GAPDH reverse, 5′-CAT GTT TGT GAT GGG CGT GAA CCA-3′ (SEQ ID NO: 6). qPCR of each sample was performed in triplicate and under the following conditions: 95° C. for 3 min followed by 40 cycles of 95° C. for 15 s and 60° C. for 1 min. The comparative 2-33Ct method was used to evaluate relative SHIP-1 mRNA levels of digested pancreatic tumors and BM cells.

### Western Blot (WB)

Single-cell suspensions of BM, digested pancreatic tumors cells and UN-KC-6141 cells treated with API (40 μM) were lysed with radioimmunoprecipitation buffer (Sigma-Aldrich), quantified with a BCA Protein Assay kit (Thermo Fisher Scientific, Waltham, MA, USA), resolved by SDS-PAGE (Thermo Fisher Scientific) and transferred onto a nitrocellulose membrane as previously described [12]. Membranes were probed with either anti-SHIP-1 (Santa Cruz Biotechnology, Dallas, TX, USA), anti-iNOS (Cell Signaling Technology) or anti-Bcl-2 (Cell Signaling Technology, Danvers, MA, USA), all at a dilution of 1:1000. All blots were re-probed with a housekeeping protein, HRP-conjugated anti-β-actin (Sigma-Aldrich), at a dilution of 1:20,000. Membranes were then probed with the respective secondary anti-mouse or rabbit IgG HRP-conjugated antibodies (1:1000) of SHIP-1 and iNOS. Detection of SHIP-1, iNOS and β-actin proteins was performed using Super Signal West Pico or Femto Chemiluminescent Substrates (Thermo Fisher Scientific) and then imaged on a Bio-Rad Chemi Doc XRS Imaging System. Normalized densitometric ratios (divided by β-actin) were determined by quantifying WB results using Image J.

### Akoya Codex

The poly-1-lysine coated coverslips containing 5-μm of Fresh Frozen (FF) tissue sections were stored at 80° C. until use. At the staining time, FF tissue sections were baked for 30 min to 1 h at 55° C. According to Akoya Biosciences protocol, the FF tissues were dewaxed, deparaffinized in xylene then rehydrated in descending ethanol concentrations (100% twice, 90%, 70%, 50%, and 30%, respectively) and washed in ddH₂O twice, each step for 5 min. Heat-induced epitope retrieval with antigen retrieval solution, pH 6, was performed using the pressure cooker at high-pressure protocol (80° C.) for 20 min. After cooling at room temperature (RT) for 30 min to 1 h, the coverslips were washed in ddH₂O twice for 2 min. Then the sample coverslips were immersed in the hydration buffer six times before being placed in the staining buffer for 20-30 min. The antibody cocktail solution was prepared to contain 1-2 μL: 200 of the antibody/sample and then added to CODEX blocking buffer (staining buffer, N blocker, G blocker, J blocker, and S blocker) to block the nonspecific binding of the antibody. For each coverslip, 190 μL of the antibody cocktail solution was added and incubated in a sealed humidity chamber for 3 h at RT or overnight at 4° C. After staining, sample coverslips were placed in the staining buffer twice for 2 min to rinse any unbound antibodies and then fixed in 1.6% paraformaldehyde diluted in the storage buffer (post-staining fixing solution) for 10 min, followed by a total of 9 quick washes in 1×PBS. After washing, the sample coverslips were incubated in 100% cold methanol for 5 min, followed by a total of nine dunks in 1×PBS. A fresh final fixative solution was prepared by diluting 20 μL of the CODEX fixative reagent in 1 mL of 1×PBS. The final fixative solution (190 μL) was added to the sample and incubated in a sealed humidity chamber at RT for 20 min, followed by nine quick washes in 1×PBS to remove the fixative reagent. Thereafter, sample coverslips were placed in a storage buffer at 4° C. for up to two weeks or further processed for imaging. At imaging time, the reporters' plate was prepared for the corresponding antibodies (one well/cycle), maintaining one dye type per cycle. The reporter stock solution was prepared for the total number of cycles. Each reporter was added (5 μL) to the corresponding cycle to create a reporter master mix per cycle, then gently mixed by pipetting before 245 μL of the mix was added into the corresponding well on the 96-well plate.

Images were collected using a KEYENCE BZ-X800 fluorescent microscope configured with 3 fluorescent channels (TxRed, Cy7, Cy5) and DAPI with 20×. Each tissue was imaged with a 20× oil immersion objective in a 5×5 tiled acquisition at 9 z-planes per tile. Images were subjected to deconvolution to remove out-of-focus light. Then the raw experiment data were transferred using CODEX Instrument Management version (CIM v1.29) software and processed using CODEX Processor version 1.7. The processed data were analyzed using the CODEX-MAV plugin in Image J. Then, nuclei segmentation was performed, followed by gating and clustering of the CD8+ T cells populations. Further analyses were performed using the Seurat package in R software (version 4.1) to generate dimensional reduction and clustering of the population of interest. The data were visualized using 2-dimensional plots (UMAP & tSNE plot).

### The Cancer Genome Atlas (TGCA) Database

miR-155 and SHIP-1 RNAseq values from PC patients were obtained from the cBioPortal for Cancer Genomics.

### Statistical Analysis

Results are presented as mean±Standard Deviation (S.D.) of all in vitro and in vivo experiments of at least three independent biological replicates. Significant differences were considered at p<0.05 when analyzed by unpaired two-tailed/tests using Prism 8 Software (GraphPad, San Diego, CA, USA).

### Conclusion

In conclusion, the pre-clinical results indicate that modulating miR-155 in PC to augment SHIP-1 expression may be a mechanism to enhance anti-tumor immunity and improve immunotherapy responses for PC. It is important to note that SHIP-1 directly and indirectly regulates multiple MDSC-TAM associated signaling events that can impact tumor outcomes and warrants further investigation. More importantly, the restoration of SHIP-1 expression is an ideal therapeutic strategy to regulate myelopoiesis and promote the development of activated tumoricidal M1 TAM for the triggering of effector CD8+ T cells to kill PC cells (FIG. 11). Thus, targeting miR-155/SHIP-1 is a new approach for adjuvant therapy for the treatment of pancreatic cancer, and its activity in other solid tumors warrants further exploration.

### Example 2—Treating Pancreatic Cancer (Prophetic)

A 45 year old male presents with lack of appetite, weight loss, and abdominal pain that radiates to the back. The patient is diagnosed with pancreatic ductal adenocarcinoma. The patient is administered a therapeutically effective amount of both apigenin and an miR-155 inhibitor for a predetermined amount of time. The patient's tumor load is decreased.

### REFERENCES

1. Rahib, L.; Smith, B. D.; Aizenberg, R.; Rosenzweig, A. B.; Fleshman, J. M.; Matrisian, L. M. Projecting cancer incidence and deaths to 2030: The unexpected burden of thyroid, liver, and pancreas cancers in the United States. Cancer Res. 2014, 74, 2913-2921.
2. Siegel, R. L.; Miller, K. D.; Fuchs, H. E.; Jemal, A. Cancer Statistics, 2021. CA Cancer J. Clin. 2021, 71, 7-33.
3. Tavakkoli, A.; Singal, A. G.; Waljee, A. K.; Elmunzer, B. J.; Pruitt, S. L.; McKey, T.; Rubenstein, J. H.; Scheiman, J. M.; Murphy, C. C. Racial Disparities and Trends in Pancreatic Cancer Incidence and Mortality in the United States. Clin. Gastroenterol. Hepatol. 2020, 18, 171-178.e110.
4. Mizrahi, J. D.; Surana, R.; Valle, J. W.; Shroff, R. T. Pancreatic cancer. Lancet 2020, 395, 2008-2020.
5. Zeng, S.; Pottler, M.; Lan, B.; Grutzmann, R.; Pilarsky, C.; Yang, H. Chemoresistance in Pancreatic Cancer. Int. J. Mol. Sci. 2019, 20, 4504.
6. Looi, C. K.; Chung, F. F.; Leong, C. O.; Wong, S. F.; Rosli, R.; Mai, C. W. Therapeutic challenges and current immunomodulatory strategies in targeting the immunosuppressive pancreatic tumor microenvironment. J. Exp. Clin. Cancer Res. 2019, 38, 162.
7. Schmiechen, Z. C.; Stromnes, I. M. Mechanisms Governing Immunotherapy Resistance in Pancreatic Ductal Adenocarcinoma. Front. Immunol. 2020, 11, 613815.
8. Padoan, A.; Plebani, M.; Basso, D. Inflammation and pancreatic cancer: Focus on metabolism, cytokines, and immunity. Int. J. Mol. Sci. 2019, 20, 676.
9. Draghiciu, O.; Lubbers, J.; Nijman, H. W.; Daemen, T. Myeloid derived suppressor cells—An overview of combat strategies to increase immunotherapy efficacy. OncoImmunology 2015, 4, e954829.
10. Porembka, M. R.; Mitchem, J. B.; Belt, B. A.; Hsieh, C.-S.; Lee, H.-M.; Herndon, J.; Gillanders, W. E.; Linehan, D. C.; Goedegebuure, P. J. C. I. Immunotherapy. Pancreatic adenocarcinoma induces bone marrow mobilization of myeloid-derived suppressor cells which promote primary tumor growth. Cancer Immunol. Immunother. 2012, 61, 1373-1385.
11. Pilon-Thomas, S.; Nelson, N.; Vohra, N.; Jerald, M.; Pendleton, L.; Szekeres, K.; Ghansah, T. Murine pancreatic adenocarcinoma dampens SHIP-1 expression and alters MDSC homeostasis and function. PLOS ONE 2011, 6, e27729.
12. Villalobos-Ayala, K.; Ortiz Rivera, I.; Alvarez, C.; Husain, K.; DeLoach, D.; Krystal, G.; Hibbs, M. L.; Jiang, K.; Ghansah, T. Apigenin Increases SHIP-1 Expression, Promotes Tumoricidal Macrophages and Anti-Tumor Immune Responses in Murine Pancreatic Cancer. Cancers 2020, 12, 3631.
13. Binnewies, M.; Roberts, E. W.; Kersten, K.; Chan, V.; Fearon, D. F.; Merad, M.; Coussens, L. M.; Gabrilovich, D. I.; Ostrand-Rosenberg, S.; Hedrick, C. C.; et al. Understanding the tumor immune microenvironment (TIME) for effective therapy. Nat. Med. 2018, 24, 541-550.
14. Mielgo, A.; Schmid, M. C. Impact of tumour associated macrophages in pancreatic cancer. BMB Rep. 2013, 46, 131-138.
15. Farajzadeh Valilou, S.; Keshavarz-Fathi, M.; Silvestris, N.; Argentiero, A.; Rezaei, N. The role of inflammatory cytokines and tumor associated macrophages (TAMs) in microenvironment of pancreatic cancer. Cytokine Growth Factor Rev. 2018, 39, 46-61.
16. Gabrilovich, D. I.; Nagaraj, S. Myeloid-derived suppressor cells as regulators of the immune system. Nat. Rev. Immunol. 2009, 9, 162-174.
17. Bartel, D. P. MicroRNAs: Genomics, biogenesis, mechanism, and function. Cell 2004, 116, 281-297.
18. Garzon, R.; Fabbri, M.; Cimmino, A.; Calin, G. A.; Croce, C. M. MicroRNA expression and function in cancer. Trends Mol. Med. 2006, 12, 580-587.
19. Gilles, M. E.; Slack, F. J. Let-7 microRNA as a potential therapeutic target with implications for immunotherapy. Expert Opin. Ther. Targets 2018, 22, 929-939.
20. Yu, Z.; Li, Y.; Fan, H.; Liu, Z.; Pestell, R. G. miRNAs regulate stem cell self-renewal and differentiation. Front. Genet. 2012, 3, 191.
21. Galasso, M.; Sandhu, S. K.; Volinia, S. MicroRNA expression signatures in solid malignancies. Cancer J. 2012, 18, 238-243.
22. Eis, P. S.; Tam, W.; Sun, L.; Chadburn, A.; Li, Z.; Gomez, M. F.; Lund, E.; Dahlberg, J. E. Accumulation of miR-155 and BIC RNA in human B cell lymphomas. Proc. Natl. Acad. Sci. USA 2005, 102, 3627-3632.
23. Hernandez, Y. G.; Lucas, A. L. MicroRNA in pancreatic ductal adenocarcinoma and its precursor lesions. World J. Gastrointest. Oncol. 2016, 8, 18-29.
24. Zhao, F.; Wei, C.; Cui, M. Y.; Xia, Q. Q.; Wang, S. B.; Zhang, Y. Prognostic value of microRNAs in pancreatic cancer: A meta-analysis. Aging (Albany N. Y.) 2020, 12, 9380-9404.
25. Rawat, M.; Kadian, K.; Gupta, Y.; Kumar, A.; Chain, P. S.; Kovbasnjuk, O.; Kumar, S.; Parasher, G. MicroRNA in pancreatic cancer: From biology to therapeutic potential. Genes 2019, 10, 752.
26. Lee, D. W.; Futami, M.; Carroll, M.; Feng, Y.; Wang, Z.; Fernandez, M.; Whichard, Z.; Chen, Y.; Kornblau, S.; Shpall, E. J.; et al. Loss of SHIP-1 protein expression in high-risk myelodysplastic syndromes is associated with miR-210 and miR-155. Oncogene 2012, 31, 4085-4094.
27. Baradaran, B.; Shahbazi, R.; Khordadmehr, M. Dysregulation of key microRNAs in pancreatic cancer development. Biomed. Pharmacother. 2019, 109, 1008-1015.
28. O'Connell, R. M.; Chaudhuri, A. A.; Rao, D. S.; Baltimore, D. Inositol phosphatase SHIP 1 is a primary target of miR-155. Proc. Natl. Acad. Sci. USA 2009, 106, 7113-7118.
29. Ghansah, T.; Paraiso, K. H.; Highfill, S.; Desponts, C.; May, S.; McIntosh, J. K.; Wang, J. W.; Ninos, J.; Brayer, J.; Cheng, F.; et al. Expansion of myeloid suppressor cells in SHIP-deficient mice represses allogeneic T cell responses. J. Immunol. 2004, 173, 7324-7330.
30. Hamilton, M. J.; Ho, V. W.; Kuroda, E.; Ruschmann, J.; Antignano, F.; Lam, V.; Krystal, G. Role of SHIP in cancer. Exp. Hematol. 2011, 39, 2-13.
31. Hibbs, M. L.; Raftery, A. L.; Tsantikos, E. Regulation of hematopoietic cell signaling by SHIP-1 inositol phosphatase: Growth factors and beyond. Growth Factors 2018, 36, 213-231.
32. Sly, L. M.; Ho, V.; Antignano, F.; Ruschmann, J.; Hamilton, M.; Lam, V.; Rauh, M. J.; Krystal, G. The role of SHIP in macrophages. Front. Biosci. 2007, 12, 2836-2848.
33. Kalesnikoff, J.; Sly, L. M.; Hughes, M. R.; Buchse, T.; Rauh, M. J.; Cao, L. P.; Lam, V.; Mui, A.; Huber, M.; Krystal, G. The role of SHIP in cytokine-induced signaling. Rev. Physiol. Biochem. Pharmacol. 2003, 149, 87-103.
34. Rauh, M. J.; Ho, V.; Pereira, C.; Sham, A.; Sly, L. M.; Lam, V.; Huxham, L.; Minchinton, A. I.; Mui, A.; Krystal, G. SHIP represses the generation of alternatively activated macrophages. Immunity 2005, 23, 361-374.
35. Ghansah, T. A novel strategy for modulation of MDSC to enhance cancer immunotherapy. Oncoimmunology 2012, 1, 984-985.
36. Silverman, D. T.; Swanson, C. A.; Gridley, G.; Wacholder, S.; Greenberg, R. S.; Brown, L. M.; Hayes, R. B.; Swanson, G. M.; Schoenberg, J. B.; Pottern, L. M.; et al. Dietary and nutritional factors and pancreatic cancer: A case-control study based on direct interviews. J. Natl. Cancer Inst. 1998, 90, 1710-1719.
37. Taylor, P. R.; Greenwald, P. Nutritional interventions in cancer prevention. J. Clin. Oncol. 2005, 23, 333-345.
38. Van den Brandt, P. A.; Goldbohm, R. A. Nutrition in the prevention of gastrointestinal cancer. Best Pract. Res. Clin. Gastroenterol. 2006, 20, 589-603.
39. Perrott, K. M.; Wiley, C. D.; Desprez, P. Y.; Campisi, J. Apigenin suppresses the senescence-associated secretory phenotype and paracrine effects on breast cancer cells. Geroscience 2017, 39, 161-173.
40. Qin, Y.; Zhao, D.; Zhou, H. G.; Wang, X. H.; Zhong, W. L.; Chen, S.; Gu, W. G.; Wang, W.; Zhang, C. H.; Liu, Y. R.; et al. Apigenin inhibits NF-kappaB and snail signaling, EMT and metastasis in human hepatocellular carcinoma. Oncotarget 2016, 7, 41421-41431.
41. Shukla, S.; Bhaskaran, N.; Babcook, M. A.; Fu, P.; Maclennan, G. T.; Gupta, S. Apigenin inhibits prostate cancer progression in TRAMP mice via targeting PI3K/Akt/FoxO pathway. Carcinogenesis 2014, 35, 452-460.
42. Pan, X.; Yang, Z.; Yang, Z.; Zhou, S.; Zhang, H.; Zang, L. Effect of apigenin on proliferation and apoptosis of human lung cancer NCI-H460 cells. Nan Fang Yi Ke Da Xue Xue Bao 2013, 33, 1137-1140.
43. Lee, Y.; Sung, B.; Kang, Y. J.; Kim, D. H.; Jang, J. Y.; Hwang, S. Y.; Kim, M.; Lim, H. S.; Yoon, J. H.; Chung, H. Y.; et al. Apigenin-induced apoptosis is enhanced by inhibition of autophagy formation in HCT116 human colon cancer cells. Int. J. Oncol. 2014, 44, 1599-1606.
44. Zhao, G.; Han, X.; Cheng, W.; Ni, J.; Zhang, Y.; Lin, J.; Song, Z. J. O. R. Apigenin inhibits proliferation and invasion, and induces apoptosis and cell cycle arrest in human melanoma cells. Oncol. Rep. 2017, 37, 2277-2285.
45. Angulo, P.; Kaushik, G.; Subramaniam, D.; Dandawate, P.; Neville, K.; Chastain, K.; Anant, S. Natural compounds targeting major cell signaling pathways: A novel paradigm for osteosarcoma therapy. J. Hematol. Oncol. 2017, 10, 10.
46. Ashrafizadeh, M.; Bakhoda, M. R.; Bahmanpour, Z.; Ilkhani, K.; Zarrabi, A.; Makvandi, P.; Khan, H.; Mazaheri, S.; Darvish, M.; Mirzaei, H. J. F.i.C. Apigenin as tumor suppressor in cancers: Biotherapeutic activity, nanodelivery, and mechanisms with emphasis on pancreatic cancer. Front. Chem. 2020, 8, 829.
47. Ujiki, M. B.; Ding, X.-Z.; Salabat, M. R.; Bentrem, D. J.; Golkar, L.; Milam, B.; Talamonti, M. S.; Bell, R. H.; Iwamura, T.; Adrian, T. E. Apigenin inhibits pancreatic cancer cell proliferation through G2/M cell cycle arrest. Mol. Cancer 2006, 5, 76.
48. Yan, X.; Qi, M.; Li, P.; Zhan, Y.; Shao, H. Apigenin in cancer therapy: Anti-cancer effects and mechanisms of action. Cell Biosci. 2017, 7, 50.
49. Ahmed, S. A.; Parama, D.; Daimari, E.; Girisa, S.; Banik, K.; Harsha, C.; Dutta, U.; Kunnumakkara, A. B. Rationalizing the therapeutic potential of apigenin against cancer. Life Sci. 2021, 267, 118814.
50. Johnson, J. L.; de Mejia, E. G. Interactions between dietary flavonoids apigenin or luteolin and chemotherapeutic drugs to potentiate anti-proliferative effect on human pancreatic cancer cells, in vitro. Food Chem. Toxicol. 2013, 60, 83-91.
51. Jang, J. Y.; Sung, B.; Kim, N. D. Role of Induced Programmed Cell Death in the Chemopreventive Potential of Apigenin. Int. J. Mol. Sci. 2022, 23, 3757.
52. Oishi, M.; izumi, Y.; Taniguchi, T.; Goi, W.; Miki, T.; Sakai, T. Apigenin sensitizes prostate cancer cells to Apo2L/TRAIL by targeting adenine nucleotide translocase-2. PLOS ONE 2013, 8, e55922.
53. Chen, M.; Wang, X.; Zha, D.; Cai, F.; Zhang, W.; He, Y.; Huang, Q.; Zhuang, H.; Hua, Z.-C. Apigenin potentiates TRAIL therapy of non-small cell lung cancer via upregulating DR4/DR5 expression in a p53-dependent manner. Sci. Rep. 2016, 6, 35468.
54. Shukla, S.; Gupta, S. Apigenin: A promising molecule for cancer prevention. Pharm. Res. 2010, 27, 962-978.
55. Nelson, N.; Szekeres, K.; Iclozan, C.; Rivera, I. O.; McGill, A.; Johnson, G.; Nwogu, O.; Ghansah, T. Apigenin: Selective CK2 inhibitor increases Ikaros expression and improves T cell homeostasis and function in murine pancreatic cancer. PLOS ONE 2017, 12, e0170197.
56. Corbett, T.; Roberts, B.; Leopold, W.; Peckham, J.; Wilkoff, L.; Griswold, D.; Schabel, F. Induction and chemotherapeutic response of two transplantable ductal adenocarcinomas of the pancreas in C57BL/6 mice. Cancer Res. 1984, 44, 717-726.
57. Torres, M. P.; Rachagani, S.; Souchek, J. J.; Mallya, K.; Johansson, S. L.; Batra, S. K. Novel pancreatic cancer cell lines derived from genetically engineered mouse models of spontaneous pancreatic adenocarcinoma: Applications in diagnosis and therapy. PLOS ONE 2013, 8, e80580.
58. Husain, K.; Francois, R. A.; Yamauchi, T.; Perez, M.; Sebti, S. M.; Malafa, M. P. Vitamin E delta-tocotrienol augments the antitumor activity of gemcitabine and suppresses constitutive NF-kappaB activation in pancreatic cancer. Mol. Cancer Ther. 2011, 10, 2363-2372.
59. Chai, M. G.; Kim-Fuchs, C.; Angst, E.; Sloan, E. K. Bioluminescent orthotopic model of pancreatic cancer progression. J. Vis. Exp. 2013, 76, e50395.
60. Tsantikos, E.; Lau, M.; Castelino, C. M.; Maxwell, M. J.; Passey, S. L.; Hansen, M. J.; McGregor, N. E.; Sims, N. A.; Steinfort, D. P.; Irving, L. B.; et al. Granulocyte-CSF links destructive inflammation and comorbidities in obstructive lung disease. J. Clin. Investig. 2018, 128, 2406-2418.
61. Liu, Y.; Chen, K.; Wang, C.; Gong, W.; Yoshimura, T.; Wang, J. M.; Liu, M. Isolation of mouse tumor-infiltrating leukocytes by percoll gradient centrifugation. Bio—Protocol 2013, 3, e892.
62. Jiang, B.-H.; Qiu, J.-G.; Wang, L.; Liu, W.-J.; Wang, J.-F.; Zhao, E.-J.; Zhou, F.-M.; Ji, X.-B.; Wang, L.-H.; Xia, Z.-K. Apigenin inhibits IL-6 transcription and suppresses esophageal carcinogenesis. Front. Pharmacol. 2019, 10, 1002.
63. Shukla, S.; Gupta, S. Apigenin-induced prostate cancer cell death is initiated by reactive oxygen species and p53 activation. Free Radic. Biol. Med. 2008, 44, 1833 1845.
64. Cao, H. H.; Chu, J. H.; Kwan, H. Y.; Su, T.; Yu, H.; Cheng, C. Y.; Fu, X. Q.; Guo, H.; Li, T.; Tse, A. K.; et al. Inhibition of the STAT3 signaling pathway contributes to apigenin-mediated anti-metastatic effect in melanoma. Sci. Rep. 2016, 6, 21731.
65. Dai, J.; Van Wie, P. G.; Fai, L. Y.; Kim, D.; Wang, L.; Poyil, P.; Luo, J.; Zhang, Z. Downregulation of NEDD9 by apigenin suppresses migration, invasion, and metastasis of colorectal cancer cells. Toxicol. Appl. Pharmacol. 2016, 311, 106-112.
66. Masuelli, L.; Benvenuto, M.; Mattera, R.; Di Stefano, E.; Zago, E.; Taffera, G.; Tresoldi, I.; Giganti, M. G.; Frajese, G. V.; Berardi, G.; et al. In Vitro and In Vivo Anti-tumoral Effects of the Flavonoid Apigenin in Malignant Mesothelioma. Front. Pharmacol. 2017, 8, 373.
67. Meng, S.; Zhu, Y.; Li, J. F.; Wang, X.; Liang, Z.; Li, S. Q.; Xu, X.; Chen, H.; Liu, B.; Zheng, X. Y.; et al. Apigenin inhibits renal cell carcinoma cell proliferation. Oncotarget 2017, 8, 19834-19842.
68. Tseng, T. H.; Chien, M. H.; Lin, W. L.; Wen, Y. C.; Chow, J. M.; Chen, C. K.; Kuo, T. C.; Lee, W. J. Inhibition of MDA-MB-231 breast cancer cell proliferation and tumor growth by apigenin through induction of G2/M arrest and histone H3 acetylation-mediated p21 (WAF1/CIP1) expression. Environ. Toxicol. 2017, 32, 434-444.
69. Qiu, W.; Su, G. H. Development of orthotopic pancreatic tumor mouse models. Methods Mol. Biol. 2013, 980, 215-223.
70. Arango, D.; Diosa-Toro, M.; Rojas-Hernandez, L. S.; Cooperstone, J. L.; Schwartz, S. J.; Mo, X.; Jiang, J.; Schmittgen, T. D.; Doseff, A. I. Dietary apigenin reduces LPS-induced expression of miR-155 restoring immune balance during inflammation. Mol. Nutr. Food Res. 2015, 59, 763-772.
71. Chumsakul, O.; Wakayama, K.; Tsuhako, A.; Baba, Y.; Takai, Y.; Kurose, T.; Honma, Y.; Watanabe, S. Apigenin Regulates Activation of Microglia and Counteracts Retinal Degeneration. J. Ocul. Pharmacol. Ther. 2020, 36, 311-319.
72. Thyagarajan, A.; Alshehri, M. S. A.; Miller, K. L.; Sherwin, C. M.; Travers, J. B.; Sahu, R. P. Myeloid-derived suppressor cells and pancreatic cancer: Implications in novel therapeutic approaches. Cancers 2019, 11, 1627.
73. Wang, C.; Yu, X.; Cao, Q.; Wang, Y.; Zheng, G.; Tan, T. K.; Zhao, H.; Zhao, Y.; Wang, Y.; Harris, D. C. Characterization of murine macrophages from bone marrow, spleen and peritoneum. BMC Immunol. 2013, 14, 6.
74. Balli, D.; Rech, A. J.; Stanger, B. Z.; Vonderheide, R. H. Immune Cytolytic Activity Stratifies Molecular Subsets of Human Pancreatic Cancer. Clin. Cancer Res. 2017, 23, 3129-3138.
75. Stromnes, I. M.; Brockenbrough, J. S.; Izeradjene, K.; Carlson, M. A.; Cuevas, C.; Simmons, R. M.; Greenberg, P. D.; Hingorani, S. R. Targeted depletion of an MDSC subset unmasks pancreatic ductal adenocarcinoma to adaptive immunity. Gut 2014, 63, 1769-1781.
76. Torphy, R. J.; Zhu, Y.; Schulick, R. D. Immunotherapy for pancreatic cancer: Barriers and breakthroughs. Ann. Gastroenterol. Surg. 2018, 2, 274-281.
77. Zhu, Y.; Knolhoff, B. L.; Meyer, M. A.; Nywening, T. M.; West, B. L.; Luo, J.; Wang-Gillam, A.; Goedegebuure, S. P.; Linehan, D. C.; DeNardo, D. G. CSF1/CSFIR blockade reprograms tumor-infiltrating macrophages and improves response to T-cell checkpoint immunotherapy in pancreatic cancer models. Cancer Res. 2014, 74, 5057-5069.
78. Balachandran, V. P.; Beatty, G. L.; Dougan, S. K. Broadening the Impact of Immunotherapy to Pancreatic Cancer: Challenges and Opportunities. Gastroenterology 2019, 156, 2056-2072.
79. Collins, M. A.; Bednar, F.; Zhang, Y.; Brisset, J.-C.; Galban, S.; Galbán, C. J.; Rakshit, S.; Flannagan, K. S.; Adsay, N. V.; di Magliano, M. P. Oncogenic Kras is required for both the initiation and maintenance of pancreatic cancer in mice. J. Clin. Investig. 2012, 122, 639-653.
80. Totiger, T. M.; Srinivasan, S.; Jala, V. R.; Lamichhane, P.; Dosch, A. R.; Gaidarski, A. A.; Joshi, C.; Rangappa, S.; Castellanos, J.; Vemula, P. K. Urolithin A, a novel natural compound to target PI3K/AKT/mTOR pathway in pancreatic cancer. Mol. Cancer Ther. 2019, 18, 301-311.
81. Mattiske, S.; Suetani, R. J.; Neilsen, P. M.; Callen, D. F. The Oncogenic Role of miR-155 in Breast Cancer. Cancer Epidemiol. Biomark. Prev. 2012, 21, 1236-1243.
82. Zadeh, M. M.; Ranji, N.; Motamed, N. Deregulation of miR-21 and miR-155 and their putative targets after silibinin treatment in T47D breast cancer cells. Iran. J. Basic Med. Sci. 2015, 18, 1209.
83. Ravishankar, D.; Rajora, A. K.; Greco, F.; Osborn, H. M. Flavonoids as prospective compounds for anti-cancer therapy. Int. J. Biochem. Cell Biol. 2013, 45, 2821-2831.
84. Volinia, S.; Calin, G. A.; Liu, C. G.; Ambs, S.; Cimmino, A.; Petrocca, F.; Visone, R.; Iorio, M.; Roldo, C.; Ferracin, M.; et al. A microRNA expression signature of human solid tumors defines cancer gene targets. Proc. Natl. Acad. Sci. USA 2006, 103, 2257-2261.
85. Cote, G. A.; Gore, A. J.; McElyea, S. D.; Heathers, L. E.; Xu, H.; Sherman, S.; Korc, M. A pilot study to develop a diagnostic test for pancreatic ductal adenocarcinoma based on differential expression of select miRNA in plasma and bile. Am. J. Gastroenterol. 2014, 109, 1942-1952.
86. Mikamori, M.; Yamada, D.; Eguchi, H.; Hasegawa, S.; Kishimoto, T.; Tomimaru, Y.; Asaoka, T.; Noda, T.; Wada, H.; Kawamoto, K. J. MicroRNA-155 controls exosome synthesis and promotes gemcitabine resistance in pancreatic ductal adenocarcinoma. Sci. Rep. 2017, 7, 42339.
87. Rakhmilevich, A. L.; Alderson, K. L.; Sondel, P. M. T-cell-independent antitumor effects of CD40 ligation. Int. Rev. Immunol. 2012, 31, 267-278.
88. Schoenberger, S. P.; Toes, R. E.; Van Der Voort, E. I.; Offringa, R.; Melief, C. J. T-cell help for cytotoxic T lymphocytes is mediated by CD40-CD40L interactions. Nature 1998, 393, 480-483.

89. J. Q.; Zeng, S.; Vitiello, G. A.; Seifert, A. M.; Medina, B. D.; Beckman, M. J.; Loo, J. K.; Santamaria-Barria, J.; Maltbaek, J. H.; Param, N. J. Macrophages and CD8+ T cells mediate the antitumor efficacy of combined CD40 ligation and imatinib therapy in gastrointestinal stromal tumors. Cancer Immunol. Res. 2018, 6, 434-447.
90. Li, L.; Zhang, J.; Diao, W.; Wang, D.; Wei, Y.; Zhang, C. Y.; Zen, K. MicroRNA-155 and MicroRNA-21 promote the expansion of functional myeloid-derived suppressor cells. J. Immunol. 2014, 192, 1034-1043.
91. Faget, J.; Peters, S.; Quantin, X.; Meylan, E.; Bonnefoy, N. Neutrophils in the era of immune checkpoint blockade. J. Immunother. Cancer 2021, 9, e002242.
92. Jin, L.; Kim, H. S.; Shi, J. Neutrophil in the Pancreatic Tumor Microenvironment. Biomolecules 2021, 11, 1170.
93. Rauh, M. J.; Sly, L. M.; Kalesnikoff, J.; Hughes, M. R.; Cao, L. P.; Lam, V.; Krystal, G. The role of SHIP 1 in macrophage programming and activation. Biochem. Soc. Trans. 2004, 32, 785-788.
94. Krystal, G.; Hamilton, M. J.; Bennewith, K. L. SHIP prevents metastasis. Aging 2016, 8, 837-838.
95. Lakhanpal, G. K.; Vecchiarelli-Federico, L. M.; Li, Y.-J.; Cui, J.-W.; Bailey, M. L.; Spaner, D. E.; Dumont, D. J.; Barber, D. L.; Ben-David, Y. The inositol phosphatase SHIP-1 is negatively regulated by Fli-1 and its loss accelerates leukemogenesis. Blood 2010, 116, 428-436.
96. Alinikula, J.; Kohonen, P.; Nera, K. P.; Lassila, O. J. Concerted action of Helios and Ikaros controls the expression of the inositol 5-phosphatase SHIP. Eur. J. Immunol. 2010, 40, 2599-2607.
97. Pauls, S. D.; Marshall, A. J. Regulation of immune cell signaling by SHIP 1: A phosphatase, scaffold protein, and potential therapeutic target. Eur. J. Immunol. 2017, 47, 932-945.
98. Singh, U. P.; Murphy, A. E.; Enos, R. T.; Shamran, H. A.; Singh, N. P.; Guan, H.; Hegde, V. L.; Fan, D.; Price, R. L.; Taub, D. D.; et al. MiR-155 deficiency protects mice from experimental colitis by reducing T helper type 1/type 17 responses. Immunology 2014, 143, 478-489.
99. Wang, D.; Tang, M.; Zong, P.; Liu, H.; Zhang, T.; Liu, Y.; Zhao, Y. MiRNA-155 regulates the Th17/Treg ratio by targeting SOCS1 in severe acute pancreatitis. Front. Physiol. 2018, 9, 686.

100. Huang, C.; Li, H.; Wu, W.; Jiang, T.; Qiu, Z. Regulation of miR-155 affects pancreatic cancer cell invasiveness and migration by modulating the STAT3 signaling pathway through SOCS1. Oncol. Rep. 2013, 30, 1223-1230.

101. Kurowska-Stolarska, M.; Alivernini, S.; Ballantine, L. E.; Asquith, D. L.; Millar, N. L.; Gilchrist, D. S.; Reilly, J.; Ierna, M.; Fraser, A. R.; Stolarski, B.; et al. MicroRNA-155 as a proinflammatory regulator in clinical and experimental arthritis. Proc. Natl. Acad. Sci. USA 2011, 108, 11193-11198.

102. Rossi, M.; Negri, E.; Lagiou, P.; Talamini, R.; Dal Maso, L.; Montella, M.; Franceschi, S.; La Vecchia, C. Flavonoids and ovarian cancer risk: A case-control study in Italy. Int. J. Cancer 2008, 123, 895-898.

103. Bosetti, C.; Spertini, L.; Parpinel, M.; Gnagnarella, P.; Lagiou, P.; Negri, E.; Franceschi, S.; Montella, M.; Peterson, J.; Dwyer, J.; et al. Flavonoids and breast cancer risk in Italy. Cancer Epidemiol. Biomark. Prev. 2005, 14, 805-808.

104. Knekt, P.; Jarvinen, R.; Seppanen, R.; Hellovaara, M.; Teppo, L.; Pukkala, E.; Aromaa, A. Dietary flavonoids and the risk of lung cancer and other malignant neoplasms. Am. J. Epidemiol. 1997, 146, 223-230.

105. Hoensch, H.; Groh, B.; Edler, L.; Kirch, W. Prospective cohort comparison of flavonoid treatment in patients with resected colorectal cancer to prevent recurrence. World J. Gastroenterol. 2008, 14, 2187-2193.

106. De Font-Reaulx Rojas, E.; Dorazco-Barragan, G. Clinical stabilization in neurodegenerative diseases: Clinical study in phase II. Rev. Neurol. 2010, 50, 520-528.

In the preceding specification, all documents, acts, or information disclosed does not constitute an admission that the document, act, or information of any combination thereof was publicly available, known to the public, part of the general knowledge in the art, or was known to be relevant to solve any problem at the time of priority

The disclosures of all publications cited above are expressly incorporated herein by reference, each in its entirety, to the same extent as if each were incorporated by reference individually.

While there has been described and illustrated specific embodiments of a method of treating cancer, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A method of treating pancreatic cancer in a patient in need thereof comprising:
administering, to the patient, a therapeutically effective amount of a therapeutic agent capable of increasing expression of SH-2 containing inositol 5′ polyphosphatase 1 (SHIP 1); and
administering, to the patient, a therapeutically effective amount of a therapeutic agent capable of inhibiting expression of miR-155;
wherein administration of both of the therapeutic agents acts synergistically to treat the pancreatic cancer by augmenting anti-tumor immune responses.

2. The method of claim 1, wherein the therapeutic agent capable of increasing expression of SHIP 1 is apigenin.

3. The method of claim 1, wherein the therapeutic agents are administered concomitantly.

4. The method of claim 1, wherein the therapeutic agents are administered at least 3 times per week.

5. A method of inducing cancer cell death comprising:
administering a therapeutically effective amount of a therapeutic agent capable of increasing expression of SH-2 containing inositol 5′ polyphosphatase 1 (SHIP 1);
wherein administration of the therapeutic agent increases apoptosis and reduces the cancer cell viability.

6. The method of claim 5, wherein the therapeutic agent capable of increasing expression of SHIP 1 is apigenin.

7. The method of claim 6, further comprising administering a therapeutically effective amount of a therapeutic agent capable of inhibiting expression of miR-155.

8. The method of claim 7, wherein administration of both of the therapeutic agents acts synergistically to induce cell death.

9. The method of claim 7, wherein the therapeutically effective agents are administered concomitantly.

10. The method of claim 7, wherein the therapeutic agents are administered at least 3 times per week.

11. A method of determining prognosis of and treating pancreatic cancer in a patient in need thereof comprising:
measuring or having measured an expression level of an miR-155 biomarker in a sample from the patient;
comparing or having compared the expression level of the miR-155 from the patient to a control sample wherein a higher differential expression of the miR-155 biomarker from the patient as compared to the control sample is indicative of a poor prognosis for the patient;
administering, to the patient, a therapeutically effective amount of a therapeutic agent capable of increasing expression of SH-2 containing inositol 5′ polyphosphatase 1 (SHIP 1); and
administering, to the patient, a therapeutically effective amount of a therapeutic agent capable of inhibiting expression of miR-155;
wherein administration of both of the therapeutic agents acts synergistically to treat the pancreatic cancer.

12. The method of claim 11, further comprising the steps of:
measuring or having measured an expression level of SH-2 containing inositol 5′ polyphosphatase 1 (SHIP 1) in a sample from the patient; and
comparing or having compared the expression level of the SHIP 1 from the patient to a control sample wherein a lower differential expression of the SHIP 1 from the patient as compared to the control sample is indicative of a poor prognosis for the patient.

13. The method of claim 12, wherein the steps of claim 12 are performed prior to administration of either of the therapeutic agents.

14. The method of claim 11, wherein the therapeutic agent capable of increasing expression of SHIP 1 is apigenin.

15. The method of claim 11, wherein the therapeutic agents are administered concomitantly.
